(19) United States
(12) Patent Application Publication
(10) Pub. No.: US 2025/0130224 A1
(43) Pub. Date: Apr. 24, 2025

(54) PHOTOREACTIVE ANTIBODY BINDING DOMAINS WITH EPITOPE TAGS FOR MULTIPLEXED ANTIBODY LABELING, DETECTION, AND PURIFICATION

(71) Applicants: THE TRUSTEES OF THE UNIVERSITY OF PENNSYLVANIA, Philadelphia, PA (US); ALPHATHERA LLC, PHILADELPHIA (PA)

(72) Inventors: Andrew TSOURKAS, Bryn Mawr, PA (US); Yi Shu, Paoli, PA (US)

(73) Assignees: THE TRUSTEES OF THE UNIVERSITY OF PENNSYLVANIA, Philadelphia, PA (US); ALPHATHERA LLC, PHILADELPHIA, PA (US)

(21) Appl. No.: 18/681,488

(22) PCT Filed: Aug. 3, 2022
(86) PCT No.: PCT/US2022/039261

§ 371 Date: Feb. 5, 2024

(51) Int. Cl. G01N 33/532; G01N 33/58; G01N 33/68

(52) U.S. Cl. CPC G01N 33/532; G01N 33/58; G01N 33/6854; G01N 2333/195; G01N 2333/745

(57) ABSTRACT

Provided are systems and methods for tagging biomolecules of interest, methods of generating tagged biomolecules, and methods of use thereof. An adapter system comprises one or more epitope tags operably linked to an antibody binding domain (AbBD), which may be a photoreactive antibody binding domain (pAbBD). A pAbBD may be used to covalently photocrosslink epitope tags to immunoglobulins or immunoglobulin fragments, which may in turn be used to label biomolecules of interest. Such epitope-tagged immunoglobulins may be used in various biological research applications, including multiplexed staining assays and tandem affinity pulldown assays.

### GOVERNMENT INTEREST STATEMENT

This invention was made with government support under EB023750 awarded by the National Institutes of Health. The government has certain rights in the invention.

### FIELD OF INVENTION

The present invention relates to systems for tagging biomolecules of interest; methods of generating tagged biomolecules; and methods of use thereof.

### BACKGROUND OF INVENTION

Epitope tagging is comparable to hoisting a prominent biochemical flag. An epitope tag is any sort of biochemical moiety that has a well-characterized, readily available detection methodology, and to which investigators can attach to other molecules to ‘flag’ them. Thus, investigators may, for example, tag an unknown biomolecule with an epitope tag, and then look for the epitope tag.

Epitope tags are widely used in biological assays to assist in protein detection and purification. Typically, in such detection and purification methods, a protein must be cloned and then genetically fused with one or more of the same type of epitope tag, or a combination or various types of epitope tags. The classic cloning and recombination methodologies are laborious and challenging.

The presence of an epitope tag, or multiple different epitope tags, allows for highly specific and sensitive detection of the protein to which it is fused, using an anti-epitope antibody. This investigative approach has enabled the detection of the epitope-protein fusions in various biological samples, including in cells, tissues, biological fluids, animals, and lysates. Epitope tags are also used in protein purification, in so-called “pulldown” assays.

Despite the widespread use of epitope tags in biological research, one significant limitation to this approach is that, as explained above, it requires that the epitope tag be genetically fused to the protein-of-interest. Generating epitope-protein fusions using recombinant genetic methods is time consuming, involved work, often taking many weeks or months to optimize. The time, difficulty, and cost of generating and testing epitope-protein fusion molecules can be prohibitive and often does not allow for the detection or isolation of naturally occurring proteins, which pushes scientists to select various alternate approaches in their research. Many such alternatives, such as secondary antibody staining, though faster and cheaper than using genetically fused epitopes, can only be used in comparatively simple experiments.

Accordingly, there is a great unmet need for ready-to-use epitope tags that can be easily attached to any “off-the-shelf” protein, such as an antibody. The present invention provides an easy method for the attachment of one or more epitope tags to “off-the-shelf” proteins (e.g., commercially available antibodies), without the need for genetic modification.

Epitope tagging is achieved through the use of antibody binding domains (AbBDs) that are linked (genetically, chemically, or enzymatically) to the epitope tag and which can be used to label an antibody, antibody fragment, or protein fused or linked to an antibody or antibody fragment. This epitope-tagged antibody can then be use to detect or isolate the desired target protein(s), based on the specificity of the antibody. AbBDs may be modified to permit ready covalent crosslinking to an antibody, antibody fragment, or protein fused or linked to an antibody or antibody fragment, such as through the introduction of photoreactive non-canonical amino acids, which provides a photoreactive antibody binding domain (pAbBD). Thus, once the AbBDs (or pAbBDs) with epitope tags are generated, they can be used to label antibodies with the epitope tags. These antibodies can then be readily used to detect or isolate any desired protein(s), based on the specificity of the antibody.

Such pAbBDs include a “photocrosslinker” that, upon mixing with a chosen protein and subsequence ultraviolet illumination forms a covalent chemical crosslink with the desired protein, e.g., an antibody, antibody fragment, or protein fused or linked to an antibody or antibody fragment. The epitope-tagged antibody can then be detected using the appropriate epitope-detection methodology.

Such an epitope-tagging method offers significant improvements over existing technologies. Traditionally, multiplexing (i.e., simultaneously detecting/purifying many different biomolecules from the same sample) requires the use of secondary antibodies that are specific for the primary antibodies, based on the host and subclass of the primary antibody. (E.g., if the primary antibody is liver-staining antibody derived from rabbit, the secondary antibody is an anti-rabbit antibody derived from a mammal other than rabbit.) However, this traditional approach seriously limits the extent of achievable multiplexing, due to the relatively limited number of available antibody subclasses.

In contrast, there is a very wide range of available epitope tags, and the diversity can be further increased by using combinations of epitopes, i.e., epitope “barcoding”. Labeling proteins using multiple epitopes can also be used to improve assay sensitivity, since it has been well-established that using multiple epitope tags in tandem significantly enhances assay sensitivity. (See Y. Li, The tandem affinity purification technology: an overview, BIOTECH. LETT. (2011) 33:1487-99.)

Finally, epitope tags can also be used to improve purification of antibody-bound antigens. In such case, the combination of multiple different epitope tags can enable tandem affinity purification methods. (In many cases, this application of epitope tagging involves the insertion of protease cleavage sites between epitope tags and the tagged protein(s).)

Having such a rapid, easy-to-use epitope tagging method would dramatically widen the possibilities for multiplexed protein detection and purification, which would significantly accelerate the pace of biological research and development.

### BRIEF SUMMARY OF INVENTION

In one aspect, provided herein are adapter systems comprising an antibody binding domain (AbBD), operably linked to one or more epitope tags. In some embodiments, the adapter system comprises an AbBD that able to specifically bind to a target-protein of interest, which may include, e.g., an immunoglobulin or an immunoglobulin fragment. An immunoglobulin or immunoglobulin of-interest may comprise a natural or recombinant IgG or IgG fragment, or a protein fused or linked to an IgG or IgG fragment. Such an IgG fragment may comprise, e.g., an F_{c}, a single-chain Fᵥ, an F_{ab}, F_{ab}′, Fᵥ, F(_{ab}′)₂, an affibody, or a monobody. In some embodiments of the adapter system, the AbBD may be fused to one or more epitope tags, chemically conjugated to one or more epitope tags, or linked to one or more epitope tags using a peptide linking module. The AbBDs may comprise a whole or fragment of Protein A, Protein G, Protein L, Protein Z, or CD4; or a subdomain thereof.

In preferred embodiments of the adapter system, the AbBDs are photoreactive antibody binding domains (pAbBDs). In such embodiments of the adapter system, the pAbBDs may comprise a recombinant protein or peptide. In an embodiment of the adapter system, the pAbBDs may comprise whole or fragment of a Protein Z. In an embodiment of the adapter system, the pAbBDs may comprise whole or fragment of a Protein G B1 domain.

In some embodiments of the adapter system comprising a pAbBD, one or more photoreactive non-canonical amino acids is incorporated into each pAbBD. The photoreactive non-canonical amino acids may be selected from the group consisting of: azido-L-phenylalanine, benzophenone-alanine, benzoylphenylalanine (BPA), L-photoleucine, L-photomethionine, 3,4-difluorophenylalanine, 4,4,4,-trifluoro-L-valine, 5-fluoro-L-tryptophan, 5,5,5,-trifluoro-L-leucine, N^{ε}-(3-amino-5-azidobenzoylcarbonyl)-L-lysine, N^{ε}-(((3-((prop-2-yn-1-yloxy)methyl)-3H-diazirine-3-yl)methoxy)carbonyl)-L-lysine, and (Se—(N-(2-(3-(but-3-yn-1-yl)-3H-diazirine-3-yl)ethyl)propionamide)-3-yl-homoselenocysteine. In preferred embodiments, the photoreactive non-canonical amino acid is benzoylphenylalanine.

In an embodiment, the pAbBD may comprise either of SEQ ID NOs: 1 or 2 having one or more photoreactive non-canonical amino acids substituted therein. In an embodiment of the adapter system, the pAbBD may comprise SEQ ID NO: 2 wherein a benzoylphenylalanine residue is substituted at A24, K28, or both. In an embodiment of the adapter system, the pAbBD may comprise SEQ ID NO: 1 wherein benzoylphenylalanine residues are substituted at Q32, F5, F13, L17, N23, K35, D36, or a combination thereof. In preferred embodiments, the pAbBD comprises SEQ ID NO: 1 wherein a benzoylphenylalanine residue is substituted at least at Q32.

In some embodiments of the adapter system, the AbBD comprises one or more chemical-linking modules. The chemical-linking modules may be selected from the group consisting of: thiol, dibenzocyclooctyne, azide, alkyne, constrained alkyne, tetrazine, transcyclooctene, norbornene, and methylcyclopropene.

In some embodiments of the adapter system, the AbBD comprise one or more peptide-linking modules. The peptide-linking modules may be selected from the group consisting of: intein, c-fos, c-jun, leucine zippers, peptide Velcro, SpyTag, SpyCatcher, sortase substrates, asparaginyl endoprotease substrates, subtiligase substrates, trypsiligase substrates, transglutaminase substrates.

In certain embodiments of the adapter system, each of the one or more epitope tags are the same type of epitope tag. In other embodiments, there is one or more different epitope tags. In some embodiments, the adapter system comprises at least two epitope tags, at least three epitope tags, at least four epitope tags, or at least five, six, seven, or more epitope tags.

In some embodiments of the adapter system, the epitope tags may be of a type selected from the group consisting of: Arg-tag, Asp-tag, AU1, AU5, B-tag, Cys-tag, E, EE-tag, E2-tag, FLAG, 3×FLAG, HA, HAT, His-tag, HSV1, KT2, Lasso Tag, Myc, NorpA, OLLAAS, Phe-tag, Protein C tag, S-tag, SpyTag, Strep I, Strep II, Tag-100-tag, T7, Universal, V5, and VSV-G. It will be readily understood to those skilled in the art that the above list is not exclusive or exhaustive and that other suitable epitope tags may be used in the systems and methods described herein.

In some embodiments, a protease cleavage site is inserted between the epitope tags and/or between the AbBD and epitope tags.

In some embodiments of the adapter system, the AbBD comprises at least one cysteine residue, and the at least one cysteine residue may be rendered photocrosslinkable using a photoactive thiol-reactive agent. The photoactive thiol-reactive agent may be a maleimide reagent. In preferred embodiments, the photoactive thiol-reactive agent is 4-N-(maleimido)benzophenone.

In another aspect, provided herein are immunoglobulin-conjugates covalently linked to one or more epitope tags. In some embodiments of the epitope-modified immunoglobulin-conjugate, the immunoglobulin-conjugate comprises whole or fragment of a natural or recombinant IgG.

In some embodiments of the epitope-modified immunoglobulin-conjugate comprising one or more epitope tags, the one or more epitope tags are covalently bound to the whole or fragment of natural or recombinant IgG using photoreactive non-canonical amino acids. The photoreactive non-canonical amino acids may be selected from the group consisting of: azido-L-phenylalanine, benzophenone-alanine, benzoylphenylalanine, L-photoleucine, L-photomethionine, 3,4-difluorophenylalanine, 4,4,4,-trifluoro-L-valine, 5-fluoro-L-tryptophan, 5,5,5,-trifluoro-L-leucine, N^{ε}-(3-amino-5-azidobenzoylcarbonyl)-L-lysine, N^{ε}-(((3-((prop-2-yn-1-yloxy)methyl)-3H-diazirine-3-yl)methoxy)carbonyl)-L-lysine, and (Se—(N-(2-(3-(but-3-yn-1-yl)-3H-diazirine-3-yl)ethyl)propionamide)-3-yl-homoselenocysteine. In preferred embodiments, the photoreactive non-canonical amino acids are benzoylphenylalanine.

In some embodiments of the epitope-modified immunoglobulin-conjugate, a protease cleavage site is operably inserted between epitope tags and/or between the immunoglobulin-conjugate and the epitope tags.

In yet another aspect, provided herein are epitope-barcoded immunoglobulin-conjugates comprising a natural or recombinant IgG or fragment thereof, covalently crosslinked to one or more epitopes tags. In some embodiments of the epitope-barcoded immunoglobulin-conjugate, each epitope tag is operably linked to a protease cleavage site inserted between the epitope tag and IgG or fragment thereof.

In still another aspect, provided herein are methods of epitope-tagging an immunoglobulin or immunoglobulin fragment comprising the steps of: providing at least one epitope tag having a crosslinker module; and crosslinking the epitope tag(s) to the immunoglobulin or immunoglobulin fragment. In some embodiments, the crosslinker module comprises a photoreactive antibody binding domain (pAbBD), a click-chemistry module, or a peptide-linking module.

In some embodiments, the immunoglobulin or immunoglobulin fragment may comprise whole or fragment of IgG, an F_{c}, a single-chain Fᵥ, an F_{ab}, F_{ab}′, Fᵥ, F(_{ab}′)₂, an affibody, monobody, anticalin, DARPin, or Knottin that has been fused or operably linked to IgG, F_{c}, or variant thereof.

In some embodiments, crosslinking the epitope tags to the immunoglobulin or immunoglobulin fragment comprises photo-crosslinking the epitope tags to the immunoglobulin or immunoglobulin fragment.

In some embodiments of the method, the epitope tags may comprise an antibody-binding domain (AbBD) selected from the group consisting of: Protein A, Protein G, Protein L, Protein Z, or CD4; or a subdomain or functional fragment thereof.

In some embodiments of the method, the AbBDs comprise photoreactive antibody-binding domains (pAbBDs) comprising one or more photoreactive non-canonical amino acids. The photoreactive non-canonical amino acids may be selected from the group consisting of: azido-L-phenylalanine, benzophenone-alanine, benzoylphenylalanine, L-photoleucine, L-photomethionine, 3,4-difluorophenylalanine, 4,4,4,-trifluoro-L-valine, 5-fluoro-L-tryptophan, 5,5,5,-trifluoro-L-leucine, N^{ε}-(3-amino-5-azidobenzoylcarbonyl)-L-lysine, N^{ε}-(((3-((prop-2-yn-1-yloxy)methyl)-3H-diazirine-3-yl)methoxy)carbonyl)-L-lysine, and (Se—(N-(2-(3-(but-3-yn-1-yl)-3H-diazirine-3-yl)ethyl)propionamide)-3-yl-homoselenocysteine. In preferred embodiments of the method, the photoreactive non-canonical amino acids are benzoylphenylalanine.

In an embodiment, the pAbBD may comprise either of SEQ ID NOs: 1 or 2, having one or more photoreactive non-canonical amino acids substituted therein. In one embodiment, the pAbBD comprises SEQ ID NO: 2 wherein a benzoylphenylalanine residue is substituted at A24, K28, or both. In still another embodiment, may comprise SEQ ID NO: 1 wherein a benzoylphenylalanine residue is substituted at Q32, F5, F13, L17, N23, K35, D36, or a combination thereof. In preferred embodiments, the pAbBD comprises SEQ ID NO: 1 wherein a benzoylphenylalanine residue is substituted at least at Q32.

In an embodiment of the method, photo-crosslinking comprises mixing the epitope tags and immunoglobulin or immunoglobulin fragment and exposing the mixture to ultraviolet light. In preferred embodiments, the ultraviolet light has a wavelength of about 365 nanometers.

In some embodiments of the method, the epitope tags further comprise one or more protease cleavage sites, such that upon crosslinking, the protease cleavage site is operably linked between the epitope tag and immunoglobulin or immunoglobulin fragment.

In some embodiments, the crosslinker module comprises an AbBD comprising at least one cysteine residue, and wherein crosslinking comprises photoactivating a photoactive thiol-reactive agent. In such embodiments, the photoactive thiol-reactive agent may be a maleimide reagent. In some such embodiments, the maleimide reagent is 4-N-(maleimido)benzophenone.

In still another aspect, provided herein are methods of immunostaining a biological sample comprising exposing the sample to at least one epitope-modified immunoglobulin-conjugate that has been previously modified in accordance with a method of epitope-tagging an immunoglobulin or immunoglobulin fragment described herein.

In some embodiments, the immunostaining methods further comprise a second step of exposing the sample to secondary-antibodies which are specific to the types of epitope tags bound to the epitope-modified immunoglobulin-conjugates.

Embodiments of the immunostaining methods may comprise exposing the sample to at least two different epitope-modified immunoglobulin-conjugates. Embodiments of the method of immunostaining may comprise exposing the sample to at least three different epitope-modified immunoglobulin-conjugates. Embodiments of the immunostaining methods may comprise exposing the sample to at least four different epitope-modified immunoglobulin-conjugates. Embodiments of the immunostaining methods may comprise exposing the sample to at least five, six, seven, or more different epitope-modified immunoglobulin-conjugates.

In another aspect, provided herein are pull-down assay methods, comprising: binding an epitope-conjugated antibody or epitope-conjugated antibody fragment to an antigen; and isolating the antigen using affinity resin designed to specifically bind an epitope on the epitope-conjugated antibody or epitope-conjugated antibody fragment.

In some embodiments, the pull-down assay method comprises the step of cleaving the epitopes from the epitope-conjugated antibody or epitope-conjugated antibody-fragment. In preferred embodiments, cleaving the epitopes is accomplished by protease cleavage.

In some embodiments, the pull-down assay method comprises the steps of: isolating the antigen using affinity resin that is designed to specifically bind a second epitope on the epitope-conjugated antibody or epitope-conjugated antibody fragment; cleaving the second epitope from the epitope-conjugated antibody or epitope-conjugated antibody-fragment; and optionally repeating (i) and (ii) with different epitopes and/or different proteases.

In yet another aspect, provided herein are extraction assay methods comprising the steps of: attaching at least two epitope tags, in series or in parallel, to an antibody or antibody fragment specific for a biomolecule to be extracted from a mixture; antibody-labeling the biomolecule to be extracted, forming a biomolecule-epitope-complex; purifying the biomolecule-epitope-complex from the mixture using an affinity pulldown specific to one of the epitopes comprising the biomolecule-epitope-complex, then cleaving the epitope used in the purification step; repeating step (b) until all epitope tags have been used in affinity pulldown and cleaved, leaving a purified biomolecule unbound to epitope tags.

In some embodiments of the extraction assay method, the at least two epitope tags are bound to one another in series. In some embodiments of the extraction assay, the at least two epitope tags are bound to one or more antibody binding domain (AbBD) at either the C-terminus, N-terminus, or both. In some embodiments of the extraction assay, the at least two epitope tags are bound at the C-terminus. Alternatively, in some embodiments, the at least two epitope tags are bound at the N-terminus. In some embodiments of the extraction assay, the at least two epitope tags are all different epitope tags. In some embodiments of the extraction assay, the epitope tags are attached using a photocrosslink to the antibody specific for the biomolecule to be extracted.

In some embodiments, the epitope tags are selected from the non-limiting group consisting of: Arg-tag, Asp-tag, AU1, AU5, B-tag, Cys-tag, E, EE-tag, E2-tag, FLAG, 3×FLAG, HA, HAT, His-tag, HSV1, KT2, Lasso Tag, Myc, NorpA, OLLAAS, Phe-tag, Protein C tag, S-tag, SpyTag, Strep I, Strep II, Tag-100-tag, T7, Universal, V5, and VSV-G.

In some embodiments, the photoreactive non-canonical amino acids are selected from the group consisting of: azido-L-phenylalanine, benzophenone-alanine, benzoylphenylalanine, L-photoleucine, L-photomethionine, 3,4-difluorophenylalanine, 4,4,4,-trifluoro-L-valine, 5-fluoro-L-tryptophan, 5,5,5,-trifluoro-L-leucine, N^{ε}-(3-amino-5-azidobenzoylcarbonyl)-L-lysine, N^{ε}-(((3-L(prop-2-yn-1-yloxy)methyl)-3H-diazirine-3-yl)methoxy)carbonyl)-L-lysine, and (Se—(N-(2-(3-(but-3-yn-1-yl)-3H-diazirine-3-yl)ethyl)propionamide)-3-yl-homoselenocysteine.

### BRIEF DESCRIPTION OF THE FIGURES

Exemplary embodiments of the invention will be better understood and readily apparent to one of ordinary skill in the art from the following written description, by way of example only, and in conjunction with the drawings, in which:

FIGS. 1A-1K are plots indicating specific binding of epitope-tagged antibodies to bovine serum albumin (BSA) and then secondarily tagged with an epitope-specific antibody, as compared against a control non-epitope-tagged anti-BSA antibody. The plots portray relative fluorescence units (RFU) at varying BSA concentrations. Indicated x-axis concentrations are model-antigen BSA in picograms/mL, which were coated on well surfaces. The coated wells were then blocked. Solutions of anti-BSA antibody, labeled with the desired [oYo-Link®]-[epitope tag], and control untagged anti-BSA antibody, were each incubated in wells to bind antibody to surface-bound BSA. (oYo-Link® (AlphaThera) is a commercially available pAbBD specific for mammalian immunoglobulin F_{c} subunit.) Finally, wells were incubated with the appropriate anti-[epitope tag] detection antibody and antigen abundance was measured with an appropriate detection assay. The y-axes depict output data in RFU, which responds to fluorescence signal intensity of analyte, and which is directly proportional to quantity of secondarily-tagged antibody present in the well sample.

More specifically, FIG. 1A depicts a plot of RFU for anti-BSA antibody-[oYo-Link®]-[His Tag] and a control anti-BSA antibody, wherein oYo-Link® is a pAbBD and HisTag is an epitope tag, and the epitope tag was detected with an anti-HisTag detection antibody.

FIG. 1B depicts a plot of RFU for anti-BSA antibody-[oYo-Link®]-[HA Tag] and a control anti-BSA antibody, wherein oYo-Link® is a pAbBD and HA Tag is an epitope tag, and the epitope tag was detected with an anti-HA Tag detection antibody.

FIG. 1C depicts a plot of RFU for anti-BSA antibody-[oYo-Link®]-[Myc Tag] and a control anti-BSA antibody, wherein oYo-Link® is a pAbBD and Myc Tag is an epitope tag, and the epitope tag was detected with an anti-Myc Tag detection antibody.

FIG. 1D depicts a plot of RFU for anti-BSA antibody-[oYo-Link®]-[FLAG Tag] and a control anti-BSA antibody, wherein oYo-Link® is a pAbBD and FLAG Tag is an epitope tag, and the epitope tag was detected with an anti-FLAG Tag detection antibody.

FIG. 1E depicts a plot of RFU for anti-BSA antibody-[oYo-Link®]-[E Tag] and a control anti-BSA antibody, wherein oYo-Link® is a pAbBD and E Tag is an epitope tag, and the epitope tag was detected with an anti-E Tag detection antibody.

FIG. 1F depicts a plot of RFU for anti-BSA antibody-[oYo-Link®]—[V5 Tag] and a control anti-BSA antibody, wherein oYo-Link® is a pAbBD and V5 Tag is an epitope tag, and the epitope tag was detected with an anti-V5 Tag detection antibody.

FIG. 1G depicts a plot of RFU for anti-BSA antibody-[oYo-Link®]—[S Tag] and a control anti-BSA antibody, wherein oYo-Link® is a pAbBD and S Tag is an epitope tag, and the epitope tag was detected with an anti-S Tag detection antibody.

FIG. 1H depicts a plot of RFU for anti-BSA antibody-[oYo-Link®]—[VSV-G Tag] and a control anti-BSA antibody, wherein oYo-Link® is a pAbBD and VSV Tag is an epitope tag, and the epitope tag was detected with an anti-VSV Tag detection antibody.

FIG. 1I depicts a plot of RFU for anti-BSA antibody-[oYo-Link®]—[NWS Tag] epitope- and a control anti-BSA antibody, wherein oYo-Link® is a pAbBD and NWS Tag is an epitope tag, and the epitope tag was detected with an anti-HisTag detection antibody.

FIG. 1J depicts a plot of RFU for anti-BSA antibody-[oYo-Link®]—[HSV Tag] and a control anti-BSA antibody, wherein oYo-Link® is a pAbBD and HSV Tag is an epitope tag, and the epitope tag was detected with an anti-HSV Tag detection antibody.

FIG. 1K depicts a plot of RFU for anti-BSA antibody-[oYo-Link®]-[AU1 Tag] and a control anti-BSA antibody, wherein oYo-Link® is a pAbBD and AU1 Tag is an epitope tag, and the epitope tag was detected with an anti-AU1 Tag detection antibody.

FIGS. 2A-2C depict detection assay experiments using ‘barcoded’ multiple-epitope-tagged antibodies, using substantially the same experimental protocol as described above for FIGS. 1A-1K. In the experiment of FIGS. 2A-2C, the experimental antibody was an anti-BSA antibody-[oYo link]-[HA]-[FLAG]-[V5] Tag, wherein oYo-Link® is a pAbBD and [HA]-[FLAG]-[V5] is three epitope tags in series. Samples were labeled with the [oYo Link]-[HA]-[FLAG]-[V5 Tag] antibody and control untagged antibody. Each were incubated in wells to bind anti-BSA antibody to surface-bound BSA. Wells were incubated with an appropriate anti-[epitope tag] detection antibody and antigen abundance was measured with an appropriate detection assay. FIG. 2A depicts the readout in RFU after secondarily incubating the [oYo Link]-[HA]-[FLAG]-[V5 Tag] and control samples with anti-HA detection antibody. FIG. 2B depicts the readout in RFU after incubating the [oYo Link]-[HA]-[FLAG]-[V5 Tag] and control samples with anti-FLAG detection antibody. FIG. 2C depicts the readout in RFU after incubating the [oYo Link]-[HA]-[FLAG]-[V5 Tag] and control samples with secondary anti-V5 detection antibody.

FIGS. 3A-3C depict the detection assay experiments using another construct of ‘barcoded’ multiple-epitope-tagged antibodies, using substantially the same experimental protocol as described above for FIGS. 1A-1K. In the experiment of FIGS. 3A-3C, the experimental antibody was an anti-BSA antibody-[oYo link]-[FLAG]-[VSVg]-[HSV] Tag. Solutions of were labeled with the [oYo link]-[FLAG]-[VSVg]-[HSV Tag] antibody and control untagged antibody. Each were incubated in wells to bind anti-BSA antibody to surface-bound BSA. Wells were incubated with an appropriate anti-[epitope tag] detection antibody and antigen abundance was measured with an appropriate detection assay. FIG. 3A depicts the readout in RFU after secondarily incubating the [oYo Link]-[FLAG]-[VSVg]-[HSV Tag] and control samples with anti-FLAG detection antibody. FIG. 3B depicts the readout in RFU after secondarily incubating the [oYo Link]-[FLAG]-[VSVg]-[HSV Tag] and control samples with anti-VSV G detection antibody. FIG. 3C depicts the readout in RFU after secondarily incubating the [oYo Link]-[FLAG]-[VSVg]-[HSV Tag] and control samples with anti-HSV Tag detection antibody.

FIG. 4 provides three-letter-abbreviated and one-letter-abbreviated amino acid sequences for various species of epitope tags.

FIG. 5 provides genera of epitope tag sequence types. For example, epitope tag sequences may be inserted only once, in a double repeat, or in a triple repeat. A “triple combination barcode” comprises three different single epitopes, operably linked together in series.

FIG. 6 provides examples of “linkers” that may be used to operably link one or more epitope tags to an immunoglobulin. Generally, a linker sequence may be inserted between an epitope tag and, for example, an immunoglobulin, AbBD, or pAbBD, or between epitope tags. The linker sequence may be a protease-cleavable sequence, such that the epitope tag can later be removed with a protease.

FIG. 7 provides single-amino-acid abbreviation amino acid sequences for various exemplary antibody binding domains (AbBDs). One or more amino acid residues may be substituted with a photoreactive non-canonical amino acid, creating a photoreactive antibody binding domain (pAbBD), which permits the use of ultraviolet to covalently photocrosslink the pAbBDs to a target immunoglobulin or immunoglobulin fragment.

FIG. 8 provides a table of exemplary substitution sites for the photoreactive non-canonical amino acid benzoylphenylalanine (BPA) on two pAbBDs. Experiments have indicated these substitution positions allow for highly reliable photocrosslinking on a mammalian IgG or mammalian IgG fragment.

FIGS. 9A-9C provide exemplary cartoon model flowcharts of a workflow of the systems and methods of the present description. FIG. 9A provides an exemplary cartoon model FLAG-tagged pAbBD covalently photocrosslinked to an immunoglobulin. The “squiggle” represents the secondary structure of an AbBD; the three-bulb structure on the right-hand side of the squiggle represents the functional group of a photoreactive non-canonical amino acid. The pAbBD is mixed with the target immunoglobulin and then exposed to UV light. The FLAG-tagged pAbBD is then covalently crosslinked to the immunoglobulin.

FIG. 9B depicts an exemplary cartoon model of an epitope-tagged immunoglobulin binding to a target protein. Upon mixing, the epitope-tagged antibody binds to the target protein.

FIG. 9C depicts an exemplary cartoon model of an anti-FLAG detection antibody binding to a FLAG-tagged-antibody, while the FLAG-tagged-antibody is bound to its specific target. The anti-FLAG antibody may be used for visual detection, e.g., for imaging, or may be used for a pulldown assay, or for other applications.

FIGS. 10A-10C depict crystal structure models of S. aureus Protein Z, with the Q32 residue (and its covalent crosslink potential) highlighted on the Protein Z α3 subunit. (See Tashiro et al. (1997). High-resolution solution NMR structure of the Z domain of staphylococcal protein A. J MOL BIO, 272 (4), 573-90.) FIG. 10A depicts a 3D model of the Protein Z tertiary crystal structure. The highlighted portion on the α3 subunit (the rightmost pictured alpha helix) represents the location of the 32nd residue, which has been experimentally shown, when substituted with benzoylphenylalanine, to reliably photocrosslink with mammalian IgG. The terminal region pictured on the bottom of the al subunit (the leftmost pictured alpha helix) is the N-terminus. FIG. 10B depicts an exemplary 3D model of the Protein Z tertiary crystal structure linked at the N-terminus to a single-epitope FLAG tag. FIG. 10C depicts an exemplary 3D model of the Protein Z tertiary crystal structure linked at the N-terminus to a [FLAG]-[VSV-G]-[HSV1] epitope ‘barcode’ tag.

FIG. 11 depicts exemplary Epitope Tag Multiplexing Data: HeLa cells were stimulated with 1 uM Camptothecin and stained with Recombinant monoclonal rabbit anti-human/mouse/rat Ezrin antibody (R&D Systems, Cat. #AB72391), following conjugation with oYo-Link® HA Tag, and Polyclonal Rabbit×human phospho-Chk2 (T68) antibody (R&D systems, Cat. #AF1626), following conjugation with oYo-Link® DYKDDDDK Tag. the HA Tag and DYKDDDDK Tag were detected using anti-HA Alex Fluor® 594 (red) and anti-FLAG Alexa Fluor® 488 (green) secondary antibodies (counterstained with DAPI, blue).

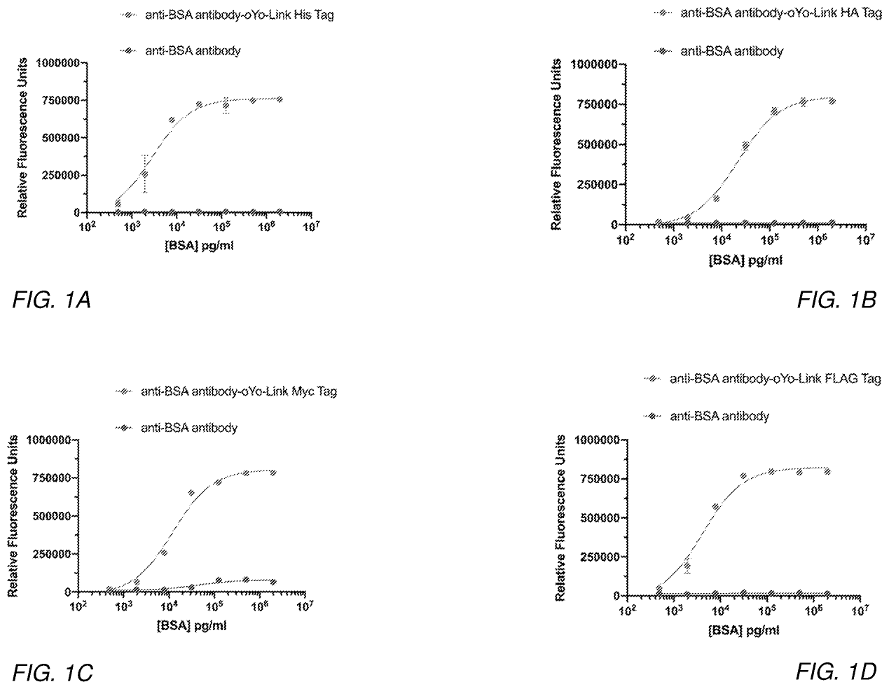

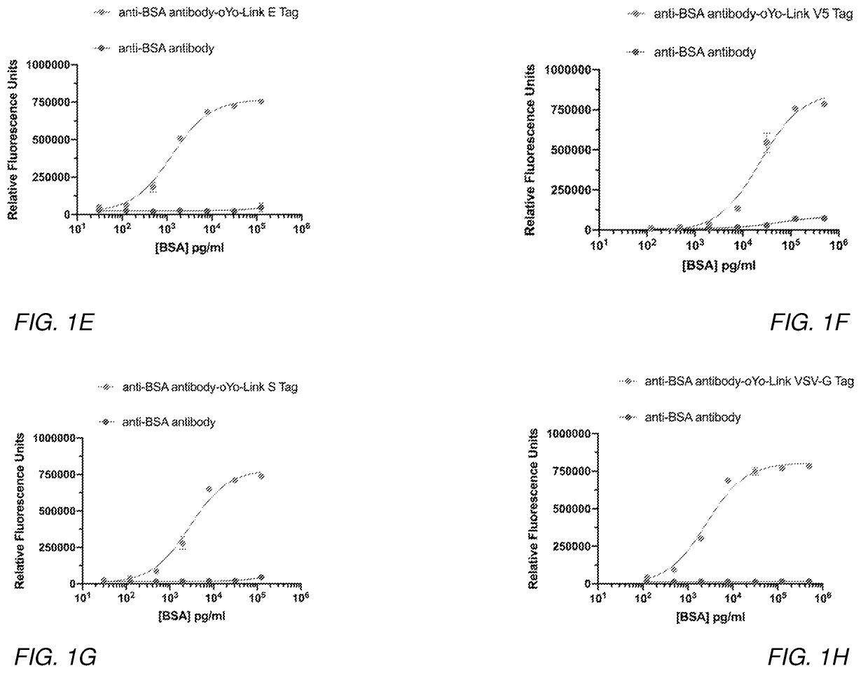

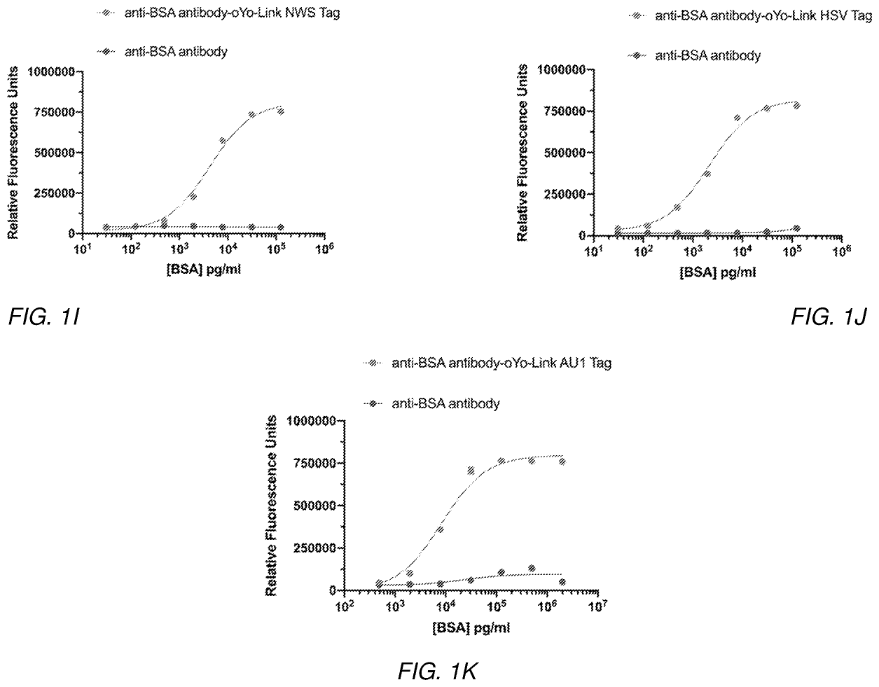

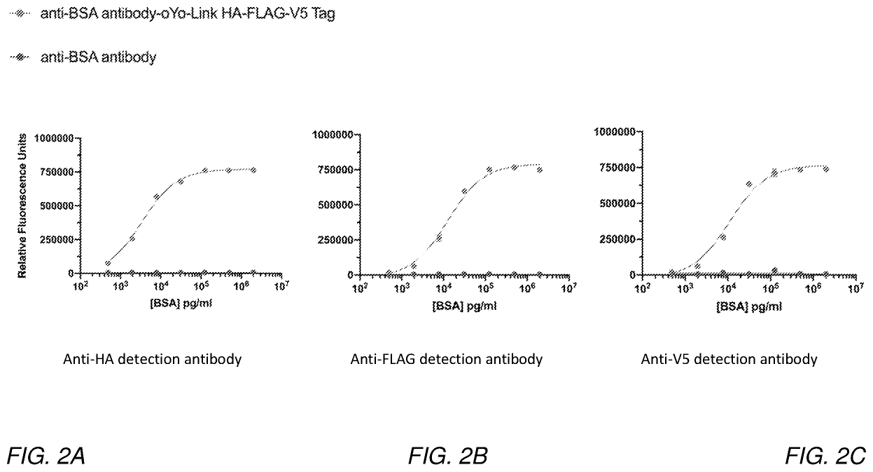

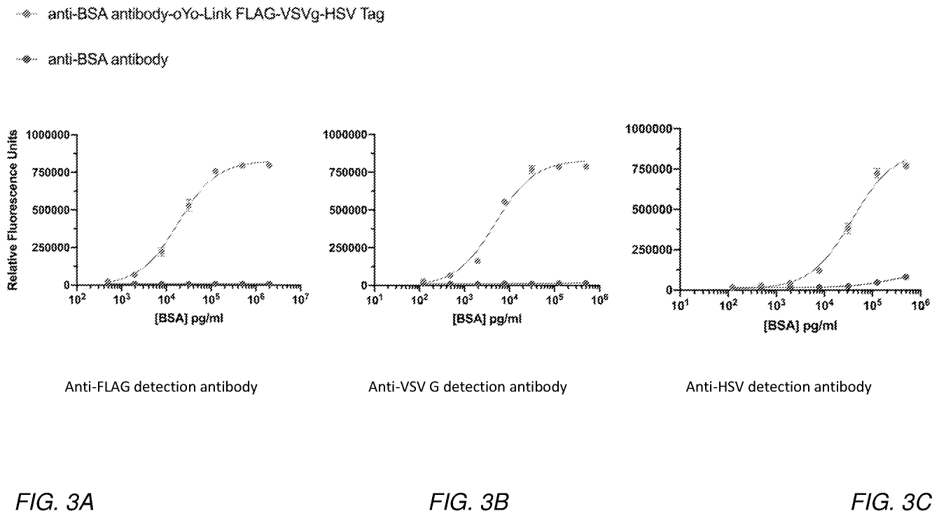

### DETAILED DESCRIPTION OF THE INVENTION

Unless otherwise defined herein, scientific and technical terms used in connection with the present application shall have the meanings that are commonly understood by those of ordinary skill in the art. Further, unless otherwise required by context, singular terms shall include pluralities and plural terms shall include the singular.

As employed above and throughout the disclosure, the following terms and abbreviations, unless otherwise indicated, shall be understood to have the following meanings:

In the present disclosure, the singular forms “a,” “an,” and “the” include the plural reference, and reference to a particular numerical value includes at least that particular value, unless the context clearly indicates otherwise. Thus, for example, a reference to “a compound” is a reference to one or more of such compounds and equivalents thereof known to those skilled in the art, and so forth. The term “plurality”, as used herein, means more than one. When a range of values is expressed, another embodiment includes from the one particular and/or to the other particular value.

Similarly, when values are expressed as approximations, by use of the antecedent “about,” it is understood that the particular value forms another embodiment. All ranges are inclusive and combinable. In the context of the present disclosure, by “about” a certain amount it is meant that the amount is within ±20% of the stated amount, or preferably within ±10% of the stated amount, or more preferably within ±5% of the stated amount.

The term “genome” refers herein to the genetic material (e.g., chromosomes) of an organism or a host cell. The term “proteome” refers herein to the entire set of proteins expressed by a genome, cell, tissue or organism. A “partial proteome” refers to a subset of the entire set of proteins expressed by a genome, cell, tissue or organism. Example “partial proteomes” include, but are not limited to, transmembrane proteins, secreted proteins, and proteins with a membrane motif.

The terms “protein,” “polypeptide,” and “peptide” refer herein to a molecule comprising amino acids joined via peptide bonds. In general “peptide” is used to refer to a sequence of 20 or less amino acids and “polypeptide” is used to refer to a sequence of greater than 20 amino acids. The term “protein of interest” refers herein to a protein encoded by a nucleic acid of interest.

The terms “synthetic polypeptide,” “synthetic peptide” and “synthetic protein” refer herein to peptides, polypeptides, and proteins that are produced by a recombinant process (i.e., expression of exogenous nucleic acid encoding the peptide, polypeptide or protein in an organism, host cell, or cell-free system) or by chemical synthesis.

The term “native” (or wild type) when used in reference to a protein refers herein to proteins encoded by the genome of a cell, tissue, or organism, other than one manipulated to produce synthetic proteins.

Abbreviations used herein for nucleotides shall adhere to industry standards as defined in WIPO Standard ST.25, Annex C, Appendix 2, Tables 1 & 2. Abbreviations used herein for the canonical proteinogenic amino acids adhere to industry standards as defined in WIPO Standard ST.25, Annex C, Appendix 2, Tables 3 & 4, and should be readily understood by persons having ordinary skill in the art. Amino acids abbreviated with the prefix D-refer to the D-enantiomer, but without any prefix shall be understood as referring to the L-enantiomer. As used herein, modified, uncommon, and non-canonical amino acids shall be abbreviated as follows: Aad=2-aminoadipic acid; bAad=3-aminoadipic acid; Acpc=1-aminocyclopropanecarboxylic acid; bAla=β-alanine (i.e., β-aminoproprionic acid); Abu=2-aminobutyric acid; 4Abu=4-aminobutyric acid (i.e., piperidinic acid); Acp=6-aminocaproic acid; Ahe=2-aminoheptanoic acid; Aib=2-aminoisobutyric acid; bAib=3-aminoisobutyric acid; AmAzZLys=N^{ε}-(3-amino-5-azidobenzoylcarbonyl)-L-lysine; Apm=2-aminopimelic acid; Bpa=benzoylphenylalanine; BPKyne=benzophenone-alanine; Dbu=2,4-diaminobutyric acid; Des=desmosine; DiZASeC=(Se—(N-(2-(3-(but-3-yn-1-yl)-3H-diazirine-3-yl)ethyl)propionamide)-3-yl-homoselenocysteine; Dpm=2,2-diaminoproprionic acid; Dpr=2,3-diaminoproprionic acid; EtGly=N-ethylglycine; EtAsn=N-ethylasparagine; Hse=homoserine (i.e., isothreonine); Hyl=hydroxylysine; aHyl=allo-hydroxylysine; 3Hyp=3-hydroxyproline; 4Hyp=4-hydroxyproline; Ide=isodesmosine; alle=allo-Isoleucine; MeGly=N-methylglycine (i.e., sarcosine); Melle=N-methylisoleucine; MeLys=6-N-methyllysine; MeVal=N-methylvaline; Nva=norvaline; Nle=norleucine; Orn=ornithine; PrDiAzK=N^{ε}-(((3-((prop-2-yn-1-yloxy)methyl)-3H-diazirine-3-yl)methoxy)carbonyl)-L-lysine. Additionally, alternative abbreviations for uncommon amino acids may be known to those having ordinary skill in the art and may be readily understood from their context. For example, sometimes norleucine may be abbreviated as “norLeu” and homoserine may be abbreviated as “homoSer.”

A “sequence read” or “read” refers to data representing a sequence of monomer units (e.g., bases) that comprise a nucleic acid molecule (e.g., DNA, cDNA, RNAs including mRNAs, rRNAs, siRNAs, miRNAs and the like). The sequence read can be measured from a given molecule via a variety of techniques.

A “mate pair” or “mated reads” or “paired-end” can refer to any two reads from a same molecule (also referred to as two arms of a same read-arm reads) that are not fully overlapped (i.e., cover different parts of the molecule). Each of the two reads would be from different parts of the same molecule, e.g., from the two ends of the molecule. As another example, one read could be for one end of the molecule in the other read for a middle part of the molecule. As a genetic sequence can be ordered from beginning to end, a first read of a molecule can be identified as existing earlier in a genome than the second read of the molecule when the first read starts and/or ends before the start and/or end of the second read. More than two reads can be obtained for each molecule, where each read would be for a different part of the molecule. Usually there is a gap (mate gap) from about 100-10,000 bases of unread sequence between two reads. Examples of mate gaps include 500+/−200 bases and 1000+/−300 bases.

“Mapping” or “aligning” refers to a process which relates a read (or a pair of reads, e.g., of a mate pair) to zero, one, or more locations in a reference sequence to which the read is similar, e.g., by matching the instantiated arm read to one or more keys within an index corresponding to a location within a reference

As used herein, an “allele” corresponds to one or more nucleotides (which may occur as a substitution or an insertion) or a deletion of one or more nucleotides. A “locus” corresponds to a location in a genome. For example, a locus can be a single base or a sequential series of bases. The term “genomic position” can refer to a particular nucleotide position in a genome or a contiguous block of nucleotide positions. A “heterozygous locus” (also called a “het”) is a location in a reference genome or a specific genome of the organism being mapped, where the copies of a chromosome do not have a same allele (e.g. a single nucleotide or a collection of nucleotides). A “het” can be a single-nucleotide polymorphism (SNP) when the locus is one nucleotide that has different alleles. A “het” can also be a location where there is an insertion or a deletion (collectively referred to as an “indel”) of one or more nucleotides or one or more tandem repeats. A single nucleotide variation (SNV) corresponds to a genomic position having a nucleotide that differs from a reference genome for a particular person. An SNV can be homozygous for a person if there is only one nucleotide at the position, and heterozygous if there are two alleles at the position. A heterozygous SNV is a het. SNP and SNV are used interchangeably herein.

Sequencing refers to the determination of intensity values corresponding to positions of one or more nucleic acids. The “intensity values” can be any signal, e.g., electrical or electromagnetic radiation, such as visible light. There can be one intensity value per base, multiple intensity values per base, or fewer intensity values than there are bases. Also, an intensity value can be for a particular position, or an intensity value can be for multiple positions of a nucleic acid. Intensity values can be restricted to predetermined values (e.g., binary or integers in a decimal numeral system), or can have continuous values.

A “sequencing process” or “sequencing run” refers to the determination of intensity values corresponding to positions of one or more nucleic acids as a batch. For example, when the sequencing involves imaging biochemical reactions of nucleic acids on a substrate, the resulting intensity values are obtained during the same sequencing run. Intensity values of nucleic acids for a different substrate would appear in different sequencing runs. A nucleic acid of a first sequencing run would not be involved in a second sequencing run (e.g., not included in a same image).

An “assumed sequence” corresponds to the sequence that is believed to be accurate. The determination may be inaccurate, but the training assumes it is accurate. The assumed sequence can be determined in a variety of ways, e.g., as described herein. An assumed sequence can include no calls, and thus an assumed sequence can have open positions between called positions.

As used herein, the term “transmembrane protein” refers to proteins that span a biological membrane. There are two basic types of transmembrane proteins. Alpha-helical proteins are present in the inner membranes of bacterial cells or the plasma membrane of eukaryotes, and sometimes in the outer membranes. Beta-barrel proteins are found only in outer membranes of Gram-negative bacteria, cell wall of Gram-positive bacteria, and outer membranes of mitochondria and chloroplasts.

As used herein, the term “external loop portion” refers to the portion of transmembrane protein that is positioned between two membrane-spanning portions of the transmembrane protein and projects outside of the membrane of a cell.

As used herein, the term “tail portion” refers to refers to an n-terminal or c-terminal portion of a transmembrane protein that terminates in the inside (“internal tail portion”) or outside (“external tail portion”) of the cell membrane.

As used herein, the term “secreted protein” refers to a protein that is secreted from a cell.

As used herein, the term “membrane motif” refers to an amino acid sequence that encodes a motif not a canonical transmembrane domain but which would be expected by its function deduced in relation to other similar proteins to be located in a cell membrane, such as those listed in the publicly available psortb database.

As used herein, the term “consensus protease cleavage site” refers to an amino acid sequence that is recognized by a protease such as trypsin or pepsin.

As used herein, the term “affinity” refers to a measure of the strength of binding between two members of a binding pair, for example, an antibody and an epitope and an epitope and an MHC-I or II haplotype.

As used herein, the term “antigen binding protein” refers to proteins that bind to a specific antigen. “Antigen binding proteins” include, but are not limited to, immunoglobulins, including polyclonal, monoclonal, chimeric, single chain, and humanized antibodies, Fab fragments, F(ab′)2 fragments, and Fab expression libraries. Various procedures known in the art are used for the production of polyclonal antibodies. For the production of antibodies, various host animals can be immunized by injection with the peptide corresponding to the desired epitope including but not limited to rabbits, mice, rats, sheep, goats, etc. Various adjuvants are used to increase the immunological response, depending on the host species, including but not limited to Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (Bacille Calmette-Guerin) and Corynebacterium parvum.

For preparation of monoclonal antibodies, any suitable technique for the production of antibody molecules by continuous cell lines in culture may be used (See, e.g., Harlow & Lane, Antibodies: A Laboratory Manual, Cold Spring Harbor Laboratory Press, Cold Spring Harbor, NY). These include, but are not limited to, the hybridoma technique originally developed by Köhler and Milstein (Nature, 256:495-497 [1975]), as well as the trioma technique, the human B-cell hybridoma technique (See, e.g., Kozbor et al., IMMUNOL. TODAY, 4:72 [1983]), and the EBV-hybridoma technique to produce human monoclonal antibodies (Cole et al., in Monoclonal Antibodies and Cancer Therapy, Alan R. Liss, Inc., pp. 77-96 [1985]). Suitable monoclonal antibodies, including recombinant chimeric monoclonal antibodies and chimeric monoclonal antibody fusion proteins may be prepared as described herein.

Techniques described for the production of single chain antibodies (U.S. Pat. No. 4,946,778) can be adapted to produce specific single chain antibodies as desired. Techniques known in the art for the construction of Fab expression libraries (See, e.g., Huse et al., SCIENCE, 246:1275-1281 [1989]) allow rapid and easy identification of monoclonal Fab fragments with the desired specificity.

Antibody fragments that contain the idiotype (antigen binding region) of the antibody molecule can be generated by known techniques. For example, such fragments include but are not limited to: the F(ab′)2 fragment that can be produced by pepsin digestion of an antibody molecule; the Fab′ fragments that can be generated by reducing the disulfide bridges of an F(ab′)2 fragment, and the Fab fragments that can be generated by treating an antibody molecule with papain and a reducing agent.

Genes encoding antigen-binding proteins can be isolated by methods known in the art. In the production of antibodies, screening for the desired antibody can be accomplished by techniques known in the art (e.g., radioimmunoassay, ELISA (enzyme-linked immunosorbant assay), “sandwich” immunoassays, immunoradiometric assays, gel diffusion precipitin reactions, immunodiffusion assays, in situ immunoassays (using colloidal gold, enzyme or radioisotope labels, for example), Western Blots, precipitation reactions, agglutination assays (e.g., gel agglutination assays, hemagglutination assays, etc.), complement fixation assays, immunofluorescence assays, protein A assays, and immunoelectrophoresis assays, etc.) etc.

As used herein, the terms “computer memory” and “computer memory device” refer to any storage media readable by a computer processor. Examples of computer memory include, but are not limited to, RAM, ROM, computer chips, digital video disc (DVDs), compact discs (CDs), hard disk drives (HDD), solid-state drives (SSD), and magnetic tape.

As used herein, the term “computer readable medium” refers to any device or system for storing and providing information (e.g., data and instructions) to a computer processor. Examples of computer readable media include, but are not limited to, DVDs, CDs, hard disk drives, magnetic tape and servers for streaming media over networks.

As used herein, the terms “processor” and “central processing unit” or “CPU” are used interchangeably and refer to a device that is able to read a program from a computer memory (e.g., ROM or other computer memory) and perform a set of steps according to the program.

A “machine-learning model” (also referred to as a model) refers to techniques that predict output base calls based on known results (training data). The known results can be an assumed sequence, which is assumed to be correct. As the model attempts to predict the results of the training data, the machine learning can be supervised learning, where the supervision comes from the training data.

A “base call” is a determination of a base at a position in a nucleic acid. A base call can be a no-call or a specified base. A base call can be made independently or as part of a combination of specified base (e.g., A/T), which can be for a same genomic position (e.g., if respective scores are close to each other) or for multiple positions. A “score” output from a machine-learning model can be used to determine a base call at a position. For example, a score can be provided for each of the bases. The determination of the base call based on the scores can be considered part of the model. Some models can provide a score, where the scores are used by a later process. Examples of a score can be a probability or a possibility. The probability scores for each of the bases would sum to a fixed number, i.e., one. The possibility scores are not required to sum to the fixed number. Each possibility score can be constrained to be between 0 and 1. The possibility scores could sum to 1, particularly if a model is trained well.

As used herein, the term “neural network” refers to various configurations of classifiers used in machine learning, including multilayered perceptrons with one or more hidden layers, support vector machines and dynamic Bayesian networks. These methods share in common the ability to be trained, the quality of their training evaluated and their ability to make either categorical classifications or of continuous numbers in a regression mode.

As used herein, the term “principal component analysis” refers to a mathematical process which reduces the dimensionality of a set of data (Wold, S., Sjorstrom, M., & Eriksson, L., Chemometrics and Intelligent Laboratory Systems 2001. 58:109-130.; Multivariate and Megavariate Data Analysis Basic Principles and Applications (Parts I&II) by L. Eriksson, E. Johansson, N. Kettaneh-Wold, & J. Trygg, 2006 2nd Ed. Umetrics Academy). Derivation of principal components is a linear transformation that locates directions of maximum variance in the original input data, and rotates the data along these axes. For n original variables, n principal components are formed as follows: The first principal component is the linear combination of the standardized original variables that has the greatest possible variance. Each subsequent principal component is the linear combination of the standardized original variables that has the greatest possible variance and is uncorrelated with all previously defined components. Further, the principal components are scale-independent in that they can be developed from different types of measurements.

The terms “dimensionality reduction” or “dimension reduction” (sometimes abbreviated “dred”) refers to the process of reducing the number of variables or features under consideration, via obtaining a set of “uncorrelated” principal variables.

As used herein, the term “vector” refers to any genetic element, such as a plasmid, phage, transposon, cosmid, chromosome, retrovirus, virion, etc., which is capable of replication when associated with the proper control elements and which can transfer gene sequences between cells. Thus, the term includes cloning and expression vehicles, as well as viral vectors.

As used herein, the term “cell culture” refers to any in vitro culture of cells. Included within this term are continuous cell lines (e.g., with an immortal phenotype), primary cell cultures, finite cell lines (e.g., non-transformed cells), and any other cell population maintained in vitro, including oocytes and embryos.

The term “isolated” when used in relation to a nucleic acid, as in “an isolated oligonucleotide” refers to a nucleic acid sequence that is identified and separated from at least one contaminant nucleic acid with which it is ordinarily associated in its natural source. Isolated nucleic acids are nucleic acids present in a form or setting that is different from that in which they are found in nature. In contrast, non-isolated nucleic acids are nucleic acids such as DNA and RNA that are found in the state in which they exist in nature.

The term “Protein Z” (abbreviated “pZ”) refers herein to the Z domain based on the B domain of Staphylococcus aureus Protein A. The amino-acid sequence of wild-type Protein Z is: VDNKFNKEQQNAFYEILHLPNLNEEQRNAFIQSLKDDPSQSANLLAEAKKLNDAQAPKMRM (SEQ ID NO: 1). Photoreactive Protein Z includes those where an amino acid in protein Z has been replaced with benzoylphenylalanine (BPA), such as F13BPA and F5BPA (see underlined amino acids in SEQ ID NO: 1). Examples of other BPA-containing Protein Z variants include, but are not limited to, Q32BPA, K35BPA, N28BPA, N23BPA, and L17BPA. Examples of Protein Z variants or mutants include, F5I, such as F5I K35BPA. The amino acid sequence of Protein Z may also include homologous, variant, and fragment sequences having Z domain function. In some embodiments, the amino acid sequence of a Protein Z variant may include an amino acid sequence which is 60%, 65%, 70%, 75%, 80%, 85%, 90%, 95%, or 99% identity to the sequence set forth in SEQ ID NO: 1.

The term “Protein G,” refers herein to a multidomain Streptococcal protein. “Protein G B1” refers herein to the B1 domain from a Streptococcal Protein G. Preferably, the Protein G B1 is a hypothermophilic variant of a B1 domain from a Streptococcal Protein G, which is designated “HTB1”. The amino acid sequence of HTB1 is: MTFKLIINGKTLKGEITIEAVDAAEAEKIFKOYANDYGIDGEWTYDDATKTFTVTE (SEQ ID NO: 2). Nine Protein G variants were successfully designed and expressed, each having an Fc-facing amino acid substituted by BPA: V21, A24, K28, 129, K31, Q32, D40, E42, W42 (see underlined amino acids in SEQ ID NO: 2). Two variants of HTB1, A24BPA and K28BPA, have been experimentally shown to allow ˜100% of all human IgG subtypes to be labeled. The amino acid sequence of HTB1 may also include homologous, variant, and fragment sequences having HTB1 domain function. In some embodiments, variants of the amino acid sequence of HTB1 may include an amino acid sequence which is 60%, 65%, 70%, 75%, 80%, 85%, 90%, 95%, or 99% identity to the sequence set forth in SEQ ID NO: 2.

The terms “in operable combination,” “in operable order,” and “operably linked” as used herein in the context of genetics refers to the direct or indirect linkage of nucleic acid sequences in such a manner that a nucleic acid molecule or complex capable of directing the transcription of a given gene and/or the synthesis of a desired protein molecule is produced. When used herein in the context of amino acids, peptides, polypeptides, proteins, glycoproteins, other biomolecules, and the like, the terms also refer to the direct or indirect linkage of agents in such a manner so that a functional combinatorial molecule or combinatorial complex is produced. “Operably linked” may suggest, but is not necessarily limited to, chemical fusion, genetic fusion, ligation, covalent binding, and non-covalent binding. “Operably linked” does not necessarily mean “directly linked”, i.e., a protein element may be “operably linked” to a molecule of interest while having an intermediate linker sequence or molecule, such as, e.g., a protease cleavage site. For example, if a protein I is bound to a protein II, which is ligated to a protein III, then protein I may be said to be operably linked to protein III.

As used herein, the term “purified” or “to purify” refers to the removal of undesired components from a sample. As used herein, the term “substantially purified” refers to molecules, either nucleic or amino acid sequences, that are removed from their natural environment, isolated or separated, and are at least 60% free, preferably 75% free, and most preferably 90% free from other components with which they are naturally associated. An “isolated polynucleotide” is therefore a substantially purified polynucleotide.

The terms “bacteria” and “bacterium” refer to prokaryotic organisms, including those within all of the phyla in the Kingdom Procaryotae. It is intended that the term encompass all microorganisms considered to be bacteria including Mycoplasma, Chlamydia, Actinomyces, Streptomyces, and Rickettsia. All forms of bacteria are included within this definition including cocci, bacilli, spirochetes, spheroplasts, protoplasts, etc. Also included within this term are prokaryotic organisms that are gram negative or gram positive. “Gram negative” and “gram positive” refer to staining patterns with the Gram-staining process that is well known in the art. (See e.g., Finegold and Martin, Diagnostic Microbiology, 6th Ed., C V Mosby St. Louis, pp. 13-15 [1982]). “Gram positive bacteria” are bacteria that retain the primary dye used in the Gram stain, causing the stained cells to appear dark blue to purple under the microscope. “Gram negative bacteria” do not retain the primary dye used in the Gram stain, but are stained by the counterstain. Thus, gram negative bacteria appear red. In some embodiments, the bacteria are those capable of causing disease (pathogens) and those that cause product degradation or spoilage.

The terms “fluorescent label”, “fluorescent tag”, and “fluorescent probe” describe a molecule or molecules that attach chemically to assist in the detection of a biomolecule such as, e.g., a protein, antibody, nucleic acid polymer, amino acid, and/or lipid. Fluorescent labels may comprise fluorescent proteins, such as, e.g., blue fluorescent proteins, cyan fluorescent proteins, green fluorescent proteins, red fluorescent proteins, and yellow fluorescent proteins. Exemplary fluorescent labels include, but are in no way limited to, Sirius, Azurite, EBFP, EBFP2, FCFP, Cerulean, CyPet, SCFP, eGFP, Emerald, Superfolder avGFP, T-Sapphire, RFP, mCherry, mOrange, mRaspberry, mRuby, FusionRed, EYFP, Topaz, Venus, Citrine, YPet, SYFP, and mAmetrine. Fluorescent labels may comprise dyes, such as the blue-fluorescent DNA stain 4,6-diamidino-2-phenylindole (DAPI). Fluorescent labels may also comprise other fluorescent biomolecule stains such as, e.g., BODIPY lipid conjugates.

As used herein, the terms “treat”, “treatment”, or “therapy” (as well as different forms thereof) refer to therapeutic treatment, including prophylactic or preventative measures, wherein the object is to prevent or slow down (lessen) an undesired physiological change associated with a disease or condition. Beneficial or desired clinical results include, but are not limited to, alleviation of symptoms, diminishment of the extent of a disease or condition, stabilization of a disease or condition (i.e., where the disease or condition does not worsen), delay or slowing of the progression of a disease or condition, amelioration or palliation of the disease or condition, and remission (whether partial or total) of the disease or condition, whether detectable or undetectable. Those in need of treatment include those already with the disease or condition as well as those prone to having the disease or condition or those in which the disease or condition is to be prevented.

The terms “subject,” “individual,” and “patient” are used interchangeably herein, and refer to an animal, for example a human, to whom treatment with a composition or formulation in accordance with the present disclosure, is provided. The term “subject” as used herein refers to human and non-human animals. The human can be any human of any age. In an embodiment, the human is an adult. In another embodiment, the human is a child. The human can be male, female, pregnant, middle-aged, adolescent, or elderly.

Conditions and disorders in a subject for which a particular drug, compound, composition, formulation (or combination thereof) is said herein to be “indicated” are not restricted to conditions and disorders for which that drug or compound or composition or formulation has been expressly approved by a regulatory authority, but also include other conditions and disorders known or reasonably believed by a physician or other health or nutritional practitioner to be amenable to treatment with that drug or compound or composition or formulation or combination thereof.

Sterile solutions can be prepared by incorporating the molecule, by itself or in combination with other active agents, in the required amount in an appropriate solvent with one or a combination of ingredients enumerated herein, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, one method of preparation is vacuum drying and freeze-drying, which yields a powder of an active ingredient plus any additional desired ingredient from a previously sterile-filtered solution thereof. The preparations for injections are processed, filled into containers such as ampoules, bags, bottles, syringes or vials, and sealed under aseptic conditions according to methods known in the art.

Further, the preparations may be packaged and sold in the form of a kit. Such articles of manufacture will preferably have labels or package inserts indicating that the associated compositions are useful for treating a subject suffering a viral infection as described herein.

The term “subject” includes mammals, e.g., humans, companion animals (e.g., dogs, cats, birds, and the like), farm animals (e.g., cows, sheep, pigs, horses, fowl, and the like) and laboratory animals (e.g., rats, mice, guinea pigs, birds, and the like). In some embodiments, the subject is male human or a female human.

In an aspect, provided herein are adapter systems comprising an antibody binding domain (AbBD), operably linked to one or more epitope tags. In some embodiments, the adapter system may comprise an AbBD is capable of specific binding to a target-protein of interest, which may include, e.g., an immunoglobulin or an immunoglobulin fragment. The adapter system may comprise an AbBD having one or more epitope tags operably linked in series at the C-terminus; operably linked in series at the N-terminus; operably linked in the middle of the AbBD sequence; or a combination thereof.

An immunoglobulin or immunoglobulin of-interest may comprise a natural or recombinant IgG or IgG fragment. Such an IgG fragment may comprise, e.g., an F_{c}, a single-chain Fᵥ, an F_{ab}, F_{ab}′, Fᵥ, F(_{ab}′)₂, an affibody, or a monobody. In some embodiments of the adapter system, the AbBD may be fused to one or more epitope tags, chemically conjugated to one or more epitope tags, or linked to one or more epitope tags using a peptide linking module. In other words, the AbBD may be covalently crosslinked to the epitope tag by various crosslinking means, including photocrosslinking, click chemistry, and enzymatically. The AbBDs may comprise a whole or fragment of Protein A, Protein G, Protein L, Protein Z, or CD4; F_{c} binding peptides; or a subdomain thereof.

Protein A, Protein G, Protein L, Protein Z, certain subdomains thereof, and certain variants thereof have been shown to have binding-specificity to immunoglobulins, such as mammalian IgG F_{c} domain and IgG F_{c}-containing fragments. See U.S. Pub. No. 2018/0344871, which is incorporated by reference in its entirety herein. Thus, epitope tags may be modified to comprise AbBDs such as that of Protein Z, and then can be linked to an immunoglobulin or immunoglobulin fragment.

In preferred embodiments of the adapter system, the AbBDs are photoreactive antibody binding domains (pAbBDs). In such embodiments of the adapter system, the pAbBDs may comprise a recombinant protein or peptide. In an embodiment of the adapter system, the pAbBDs may comprise whole or fragment of Protein Z. In an embodiment of the adapter system, the pAbBDs may comprise whole or fragment of a Protein G B1 domain. The pAbBDs enable photocrosslinking of epitope tags to one or more target immunoglobulins and/or immunoglobulin fragments.

In some embodiments of the adapter system comprising a pAbBD, one or more photoreactive non-canonical amino acids may be incorporated into each pAbBD. The photoreactive non-canonical amino acids may be selected from the group consisting of: azido-L-phenylalanine, benzophenone-alanine, benzoylphenylalanine, L-photoleucine, L-photomethionine, 3,4-difluorophenylalanine, 4,4,4,-trifluoro-L-valine, 5-fluoro-L-tryptophan, 5,5,5,-trifluoro-L-leucine, N^{ε}-(3-amino-5-azidobenzoylcarbonyl)-L-lysine, N^{ε}-(((3-((prop-2-yn-1-yloxy)methyl)-3H-diazirine-3-yl)methoxy)carbonyl)-L-lysine, and (Se—(N-(2-(3-(but-3-yn-1-yl)-3H-diazirine-3-yl)ethyl)propionamide)-3-yl-homoselenocysteine. In preferred embodiments, the photoreactive non-canonical amino acid is benzoylphenylalanine. Such photoreactive non-canonical amino acids and their properties are explored in Jan-Erik Hoffmann, Bifunctional Non-Canonical Amino Acids: Combining Photo-Crosslinking with Click Chemistry, BIOMOL. 10, 578 (April 2020), which is incorporated by reference in its entirety herein.

These photo-reactive non-canonical amino acids have the following chemical structures:

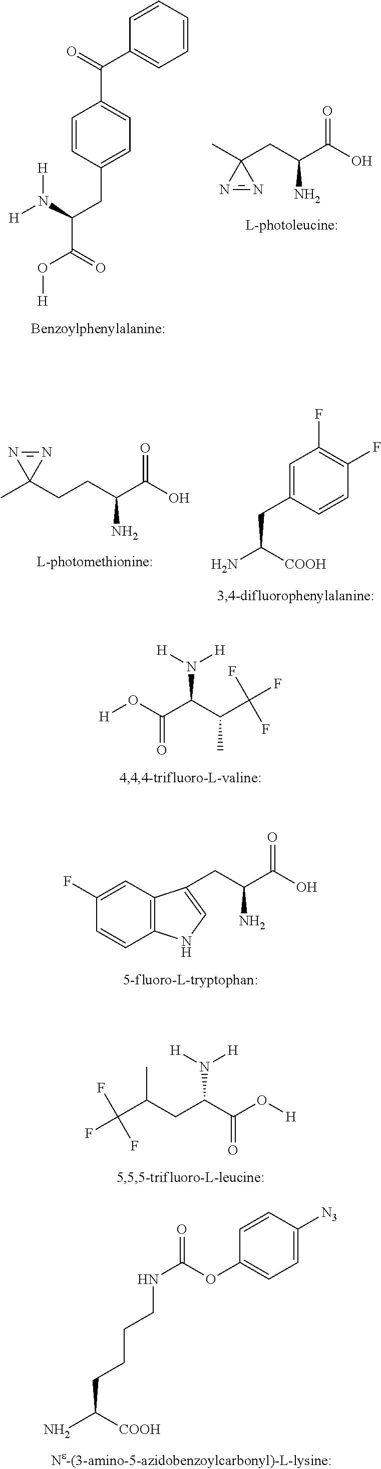

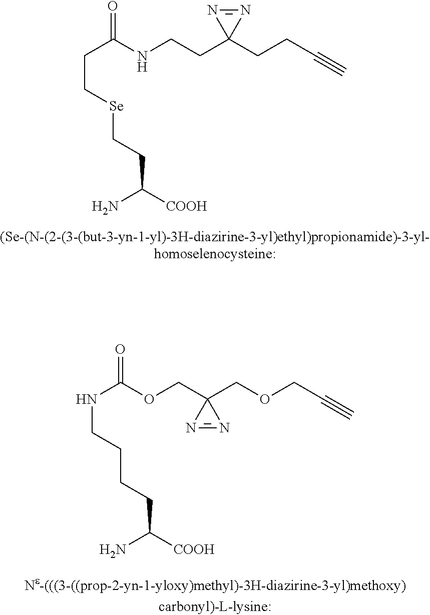 [SMILES: C#CCCC1(CCNC(=O)CC[Se]CC[C@H](N)C(=O)O)N=N1 ; C#CCOCC1(COC(=O)NCCCC[C@H](N)C(=O)O)N=N1]

It will be readily understood to those skilled in the art that the above amino acid structures imply a carboxylic acid that may exist in either bound or acid form. Further, these chemical structures suggest various closely-related homologs that may have substantially similar chemical and/or biological properties known to persons skilled in the art.

In an embodiment, the pAbBD may comprise either of SEQ ID NOs: 1 or 2, having one or more photoreactive non-canonical amino acids substituted therein. In one embodiment, the pAbBD comprises SEQ ID NO: 2, wherein a benzoylphenylalanine residue is substituted at A24, K28, or both. In one embodiment, the pAbBD comprises SEQ ID NO: 1, wherein a benzoylphenylalanine residue is substituted at least at Q32.

In some embodiments, the AbBD comprises one or more chemical-linking modules. The chemical-linking modules may be selected from the group consisting of: thiol, dibenzocyclooctyne, azide, alkyne, constrained alkyne, tetrazine, transcyclooctene, norbornene, and methylcyclopropene.

In some embodiments, the AbBD comprises one or more peptide-linking modules. The peptide-linking modules may be selected from the list consisting of: intein, c-fos, c-jun, leucine zippers, peptide Velcro, SpyTag, SpyCatcher, sortase substrates, asparaginyl endoprotease substrates, subtiligase substrates, trypsiligase substrates, transglutaminase substrates.

In some embodiments of the adapter system, there are two or more epitope tags that are the same type of epitope tag. In other embodiments, there is one or more different epitope tag. In some embodiments of the adapter system, the adapter system comprises at least two different epitope tags. In some embodiments, the adapter system comprises at least two epitope tags, at least three epitope tags, at least four epitope tags, or at least five, six, seven, or more.

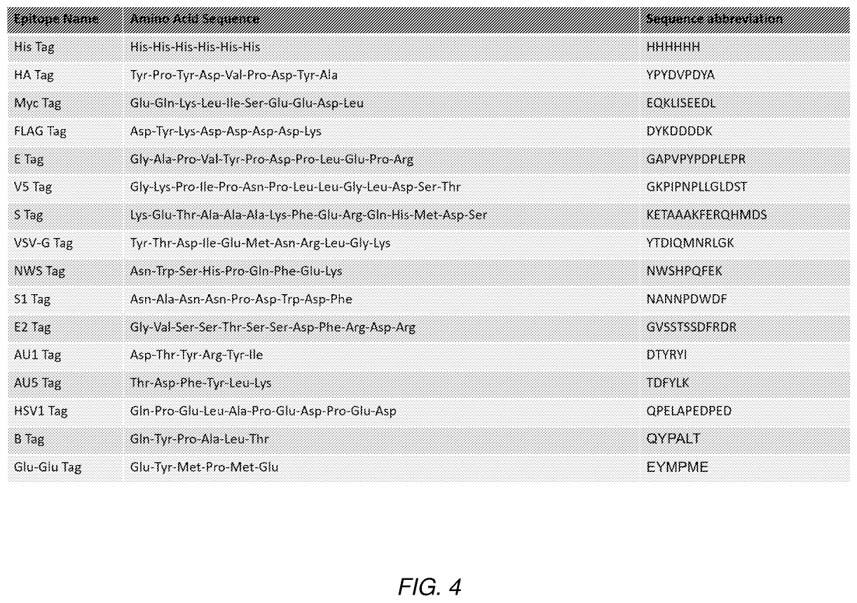

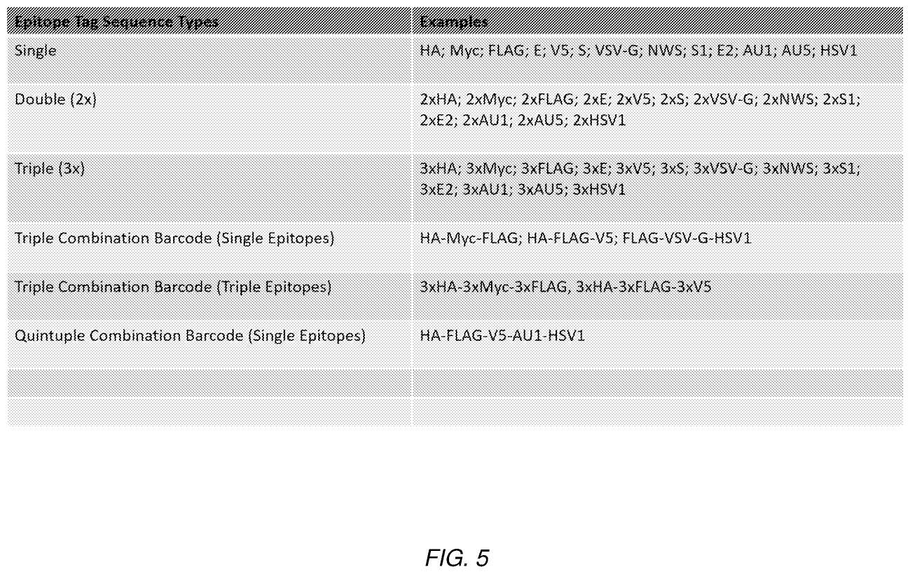

In some embodiments of the adapter system, the epitope tags may be of a type selected from the group consisting of: Arg-tag, Asp-tag, AU1, AU5, B-tag, Cys-tag, E, EE-tag, E2-tag, FLAG, 3×FLAG, HA, HAT, His-tag, HSV1, KT2, Lasso Tag, Myc, NorpA, OLLAAS, Phe-tag, Protein C tag, S-tag, SpyTag, Strep I, Strep II, Tag-100-tag, T7, Universal, V5, and VSV-G. Some non-limiting examples of epitope tag sequences are provided in FIG. 4. It will be readily understood to those skilled in the art that the above list is not exclusive or exhaustive and that other suitable epitope tags may be used in the systems and methods described herein.

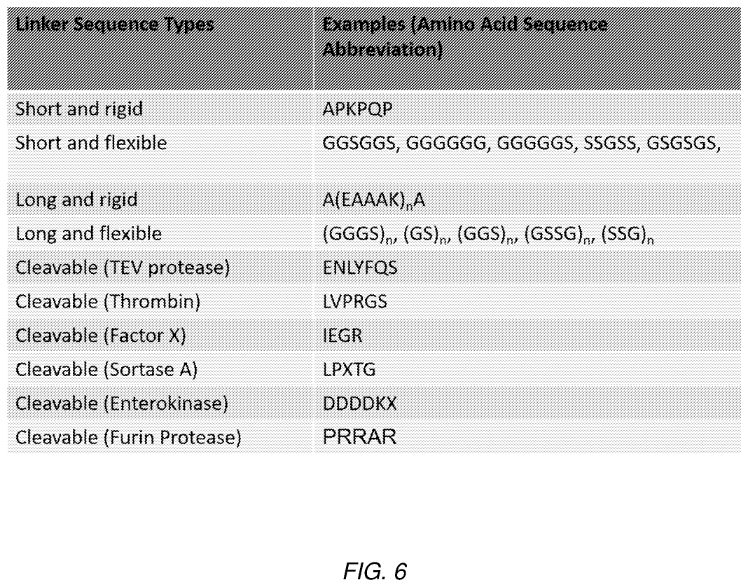

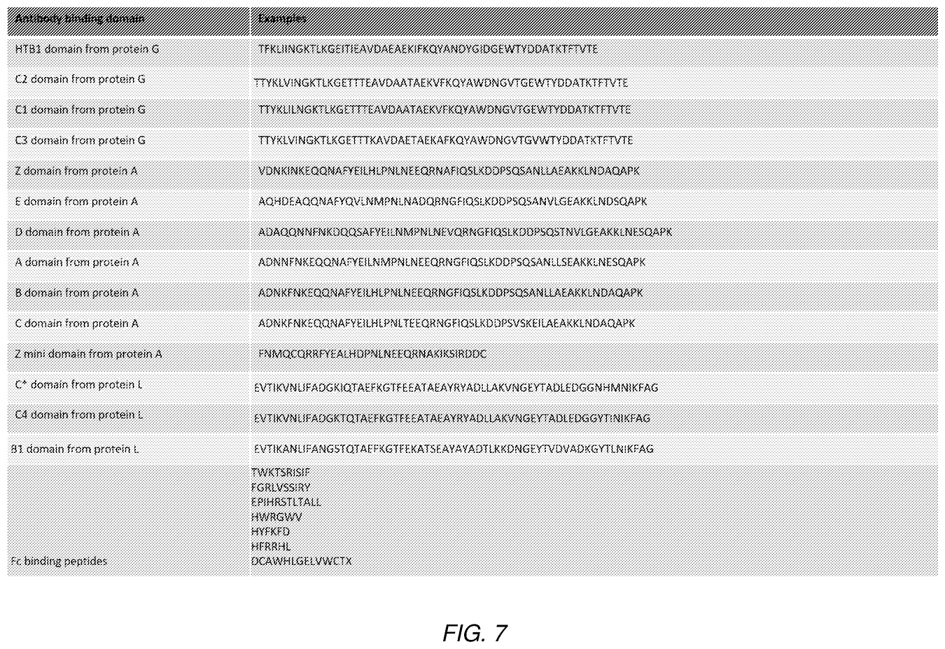

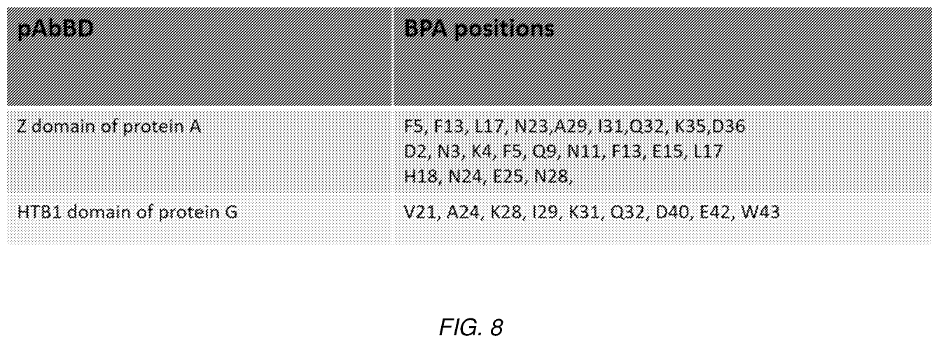

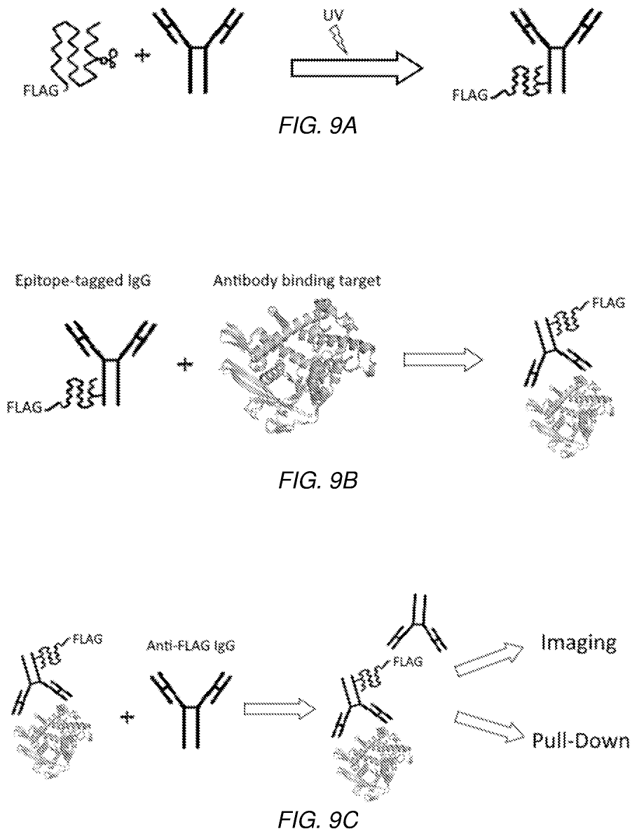

In some embodiments, a linker is inserted between the AbBD and the epitope tags, and/or between epitope tags. In some embodiments, the linker comprises a protease cleavage site. Some non-limiting examples of linker sequences are provided in FIG. 6.

In some embodiments, the AbBD may comprise at least one cysteine residue, and the at least one cysteine residue is rendered photocrosslinkable using a photoactive thiol-reactive agent. The photoactive thiol-reactive agent may be a maleimide reagent. In preferred embodiments, the photoactive thiol-reactive agent is 4-N-(maleimido)benzophenone, which has the structure:

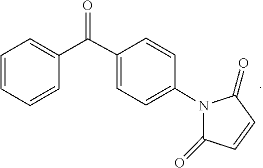 [SMILES: O=C(c1ccccc1)c1ccc(N2C(=O)C=CC2=O)cc1]

In another aspect, provided herein are immunoglobulin-conjugates covalently linked to one or more epitope tags. In some embodiments of the epitope-modified immunoglobulin-conjugate, the immunoglobulin-conjugate may comprise whole or fragment of a natural or recombinant IgG.

In some embodiments, the epitope-modified immunoglobulin-conjugate comprising one or more epitope tags, the one or more epitope tags are covalently bound to the whole or fragment of natural or recombinant IgG using photoreactive non-canonical amino acids. The photoreactive non-canonical amino acids may be selected from the group consisting of: azido-L-phenylalanine, benzophenone-alanine, benzoylphenylalanine, L-photoleucine, L-photomethionine, 3,4-difluorophenylalanine, 4,4,4,-trifluoro-L-valine, 5-fluoro-L-tryptophan, 5,5,5,-trifluoro-L-leucine, N^{ε}-(3-amino-5-azidobenzoylcarbonyl)-L-lysine, N^{ε}-(((3-((prop-2-yn-1-yloxy)methyl)-3H-diazirine-3-yl)methoxy)carbonyl)-L-lysine, and (Se—(N-(2-(3-(but-3-yn-1-yl)-3H-diazirine-3-yl)ethyl)propionamide)-3-yl-homoselenocysteine. In preferred embodiments, the photoreactive non-canonical amino acids are benzoylphenylalanine.

In some embodiments of the epitope-modified immunoglobulin-conjugate, a protease cleavage site is operably inserted between epitope tags and/or between the immunoglobulin-conjugate and the epitope tags.

In yet another aspect, provided herein are epitope-barcoded immunoglobulin-conjugates comprising a natural or recombinant IgG or fragment thereof, covalently crosslinked to one or more epitopes tags. In some embodiments, each epitope tag is operably linked to a protease cleavage site inserted between the epitope tags, or between epitope tags, and IgG or fragment thereof.

In still another aspect, provided herein are methods of epitope-tagging an immunoglobulin or immunoglobulin fragment comprising the steps of: providing at least one epitope tag having a crosslinker module; and crosslinking the epitope tags to the immunoglobulin or immunoglobulin fragment. In some embodiments, the crosslinker module comprises a photoreactive antibody binding domain (pAbBD).

In some embodiments, the immunoglobulin or immunoglobulin fragment comprises whole or fragment of IgG, an F_{c}, a single-chain Fᵥ, an F_{ab}, F_{ab}′, Fᵥ, F(_{ab}′)₂, an affibody, monobody, anticalin, DARPin, or Knottin that has been fused or operably linked to IgG, F_{c}, or variant thereof.

In some embodiments, crosslinking the epitope tags to the immunoglobulin or immunoglobulin fragment comprises photo-crosslinking the epitope tags to the immunoglobulin or immunoglobulin fragment.

In some embodiments of the method, the epitope tags comprise an antibody-binding domain (AbBD) selected from the group consisting of: Protein A, Protein G, Protein L, Protein Z, CD4; F_{c} binding peptide; or a subdomain or functional fragment thereof.

In some embodiments of the method, the AbBDs may comprise photoreactive antibody-binding domains (pAbBDs) comprising one or more photoreactive non-canonical amino acids. The photoreactive non-canonical amino acids may be selected from the group consisting of: azido-L-phenylalanine, benzophenone-alanine, benzoylphenylalanine, L-photoleucine, L-photomethionine, 3,4-difluorophenylalanine, 4,4,4,-trifluoro-L-valine, 5-fluoro-L-tryptophan, 5,5,5,-trifluoro-L-leucine, N^{ε}-(3-amino-5-azidobenzoylcarbonyl)-L-lysine, N^{ε}-(((3-((prop-2-yn-1-yloxy)methyl)-3H-diazirine-3-yl)methoxy)carbonyl)-L-lysine, and (Se—(N-(2-(3-(but-3-yn-1-yl)-3H-diazirine-3-yl)ethyl)propionamide)-3-yl-homoselenocysteine. In preferred embodiments of the method, the photoreactive non-canonical amino acids are benzoylphenylalanine.

In an embodiment, the pAbBD may comprise either of SEQ ID NOs: 1 or 2, having one or more photoreactive non-canonical amino acids substituted therein. In another embodiment, the pAbBD may comprise SEQ ID NO: 2, wherein a benzoylphenylalanine residue is substituted at A24, K28, or both. In another embodiment, the pAbBD may comprise SEQ ID NO: 1, wherein a benzoylphenylalanine residue is substituted at Q32, F5, F13, L17, N23, K35, D36, or a combination thereof. In preferred embodiments, the pAbBD comprises SEQ ID NO: 1, wherein a benzoylphenylalanine residue is substituted at least at Q32.

In some embodiments, photo-crosslinking comprises mixing the epitope tags and immunoglobulin or immunoglobulin fragment and exposing the mixture to ultraviolet light. In preferred embodiments, the ultraviolet light has a wavelength of about 365 nanometers.

In some embodiments of the method, the epitope tags further comprise one or more protease cleavage sites, such that upon crosslinking, the protease cleavage site is operably linked between the epitope tags, or between epitope tags and immunoglobulin or immunoglobulin fragment.

In some embodiments of the method, the crosslinker module comprises an AbBD comprising at least one cysteine residue, and wherein crosslinking comprises photoactivating a photoactive thiol-reactive agent. In such embodiments, the photoactive thiol-reactive agent may be a maleimide reagent. For example, the maleimide reagent is 4-N-(maleimido)benzophenone.

In still another aspect, provided herein are methods of immunostaining a biological sample comprising exposing the sample to at least one epitope-modified immunoglobulin-conjugate that has been previously modified in accordance with a method of epitope-tagging an immunoglobulin or immunoglobulin fragment described herein.

Embodiments of the methods of immunostaining may further comprise a second step of exposing the sample to secondary-antibodies which are specific to the types of epitope tags bound to the epitope-modified immunoglobulin-conjugates.

Embodiments of the methods of immunostaining may comprise exposing the sample to at least two different epitope-modified immunoglobulin-conjugates. Embodiments of the methods of immunostaining may comprise exposing the sample to at least three different epitope-modified immunoglobulin-conjugates. Embodiments of the methods of immunostaining may comprise exposing the sample to at least four different epitope-modified immunoglobulin-conjugates. Embodiments of the methods of immunostaining may comprise exposing the sample to at least five, six, seven, or more different epitope-modified immunoglobulin-conjugates.

In another aspect, provided herein are pull-down assay methods comprising: binding an epitope-conjugated antibody or epitope-conjugated antibody fragment to an antigen; and isolating the antigen using an affinity resin designed to specifically bind an epitope on the epitope-conjugated antibody or epitope-conjugated antibody fragment.

In some embodiments, the pull-down assay methods comprise the step of: cleaving the epitopes from the epitope-conjugated antibody or epitope-conjugated antibody-fragment. In preferred embodiments, cleaving the epitopes is accomplished by protease cleavage.

In some embodiments, the pull-down assay methods comprise the steps of: isolating the antigen using an affinity resin that is designed to specifically bind a second epitope on the epitope-conjugated antibody or epitope-conjugated antibody fragment; cleaving the second epitope from the epitope-conjugated antibody or epitope-conjugated antibody-fragment; and optionally repeating (i) and (ii) with different epitopes and/or proteases.

In yet another aspect, provided herein are extraction assay methods comprising the steps of: attaching at least two epitope tags, in series or in parallel, to an antibody specific for a biomolecule to be extracted from a mixture; antibody-labeling the biomolecule to be extracted, forming a biomolecule-epitope-complex; purifying the biomolecule-epitope-complex from the mixture using an affinity pulldown specific to one of the epitopes comprising the biomolecule-epitope-complex, then cleaving the epitope used in the purification step; repeating step (b) until all epitope tags have been used in affinity pulldown and cleaved, leaving a purified biomolecule unbound to epitope tags. In some embodiments, the at least two epitope tags are bound to one another in series.

In some embodiments of the extraction assay, the at least two epitope tags are bound to one or more antibody binding domain (AbBD) at either the C-terminus, N-terminus, or both. In some embodiments, the at least two epitope tags are bound at the C-terminus. Alternatively, in some embodiments of the extraction assay the at least two epitope tags are bound at the N-terminus.

In some embodiments, each of the at least two epitope tags is a different epitope tag.

In some embodiments of the extraction assay, the epitope tags are attached using a photocrosslink to the antibody specific for the biomolecule to be extracted.

In some embodiments, the epitope tags are selected from the non-limiting group consisting of: Arg-tag, Asp-tag, AU1, AU5, B-tag, Cys-tag, E, EE-tag, E2-tag, FLAG, 3×FLAG, HA, HAT, His-tag, HSV1, KT2, Lasso Tag, Myc, NorpA, OLLAAS, Phe-tag, Protein C tag, S-tag, SpyTag, Strep I, Strep II, Tag-100-tag, T7, Universal, V5, and VSV-G.

In some embodiments, the photoreactive non-canonical amino acids are selected from the group consisting of: azido-L-phenylalanine, benzophenone-alanine, benzoylphenylalanine, L-photoleucine, L-photomethionine, 3,4-difluorophenylalanine, 4,4,4,-trifluoro-L-valine, 5-fluoro-L-tryptophan, 5,5,5,-trifluoro-L-leucine, N^{ε}-(3-amino-5-azidobenzoylcarbonyl)-L-lysine, N^{ε}-(((3-((prop-2-yn-1-yloxy)methyl)-3H-diazirine-3-yl)methoxy)carbonyl)-L-lysine, and (Se—(N-(2-(3-(but-3-yn-1-yl)-3H-diazirine-3-yl)ethyl)propionamide)-3-yl-homoselenocysteine.

### EXAMPLES

Experiments demonstrating the efficacy of the epitope-tagging methods and functionality of the epitope-tagged immunoglobulins were conducted using two methodological strategies.

### Example 1

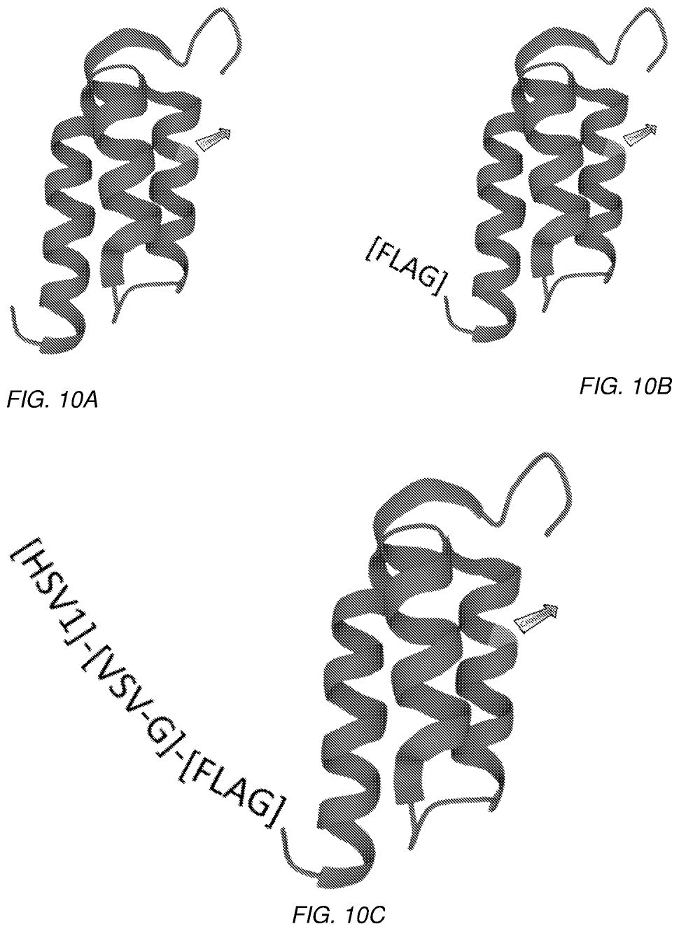

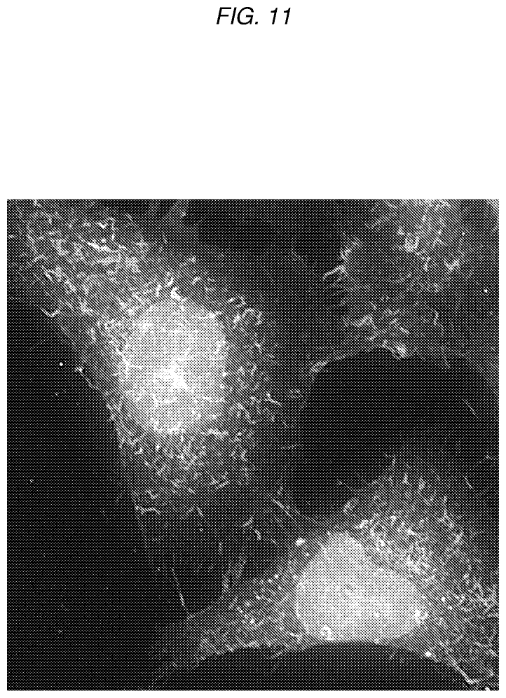

In the first experimental strategy, output was measured using horseradish peroxidase (HRP) activity. Experimental microplate wells were coated with various concentrations of bovine serum albumin (BSA) and allowed to incubate overnight. The experimental antibody construct was then created by covalently crosslinking the desired epitope to anti-BSA antibody using oYo-Link®. The tested single-epitope constructs were: (a) antibody-[oYo-Link®]-[His Tag]; (b) antibody-[oYo-Link®]-[HA Tag]; (c) antibody-[oYo-Link®]-[Myc Tag]; (d) antibody-[oYo-Link®]-[FLAG Tag]; (e) antibody-[oYo-Link®]-[E Tag]; (f) antibody-[oYo-Link®]—[V5 Tag]; (g) antibody-[oYo-Link®]—[S Tag]; (h) antibody-[oYo-Link®]—[VSV-G Tag]; (i) antibody-[oYo-Link®]—[NWS Tag]; (j) antibody-[oYo-Link®]—[HSV Tag]; and (k) antibody-[oYo-Link®]-[AU1 Tag]. The tested epitope-barcode constructs were: (a) antibody-[oYo link]-[HA]-[FLAG]-[V5 Tag]; and (b) antibody-[oYo link]-[FLAG]-[VSVg]-[HSV Tag]. FIG. 10C. The BSA-coated wells were blocked using ThermoFischer blocking buffer. Wells were then incubated with 2 μg/mL of either experimental construct or a negative-control anti-BSA antibody lacking any epitope tag. Next, the wells were incubated with 24 μg/mL of anti-[epitope tag] antibody conjugated the HRP. Activity was then measured with an HRP substrate kit.

### Example 2

The second experimental strategy used biotinylated anti-[epitope tag] antibody with a [oYo-Link®] single biotin. oYo-Link® (AlphaThera) is a commercially available pAbBD specific for mammalian immunoglobulin F_{c} subunit. Microplate wells were coated with BSA overnight. Then epitope tags were crosslinked using oYo-Link® to anti-BSA antibody and to anti-[epitope tag] antibodies labeled with oYo-Link® Single Biotin. Wells were blocked using ThermoFischer blocking buffer. Wells were then incubated with 2 μg/mL of either experimental construct or a negative-control anti-BSA antibody crosslinked to an “incorrect” epitope tag, in this case [oYo-Link®]-[His Tag]. Wells were then incubated with 2 μg/mL of biotinylated anti-[epitope tag] antibody. Next, the wells were incubated with 0.5 μg/mL of streptavidin HRP. HRP activity was measured using a substrate kit.

What is claimed:

1. An adapter system comprising an antibody binding domain (AbBD), operably linked to one or more epitope tags.

2. The adapter system of claim 1, wherein the AbBD is capable of specific binding to an immunoglobulin or an immunoglobulin fragment.

3. The adapter system of claim 2, wherein the immunoglobulin or an immunoglobulin fragment comprises a natural or recombinant IgG or IgG fragment.

4. The adapter system of claim 1, wherein the AbBD is fused to one or more epitope tags, chemically conjugated to one or more epitope tags, or linked to one or more epitope tags using a peptide linking module.

5. The adapter system of claim 1, wherein the AbBDs are photoreactive antibody binding domains (pAbBDs).

6. The adapter system of claim 5, wherein the pAbBDs comprises a recombinant protein or peptide.

7. The adapter system of claim 4, wherein the AbBDs comprise an F_{c}-binding peptide or a whole or fragment of: Protein A, Protein G, Protein L, Protein Z, or CD4; or a subdomain thereof.

8. The adapter system of claim 5, wherein the pAbBDs comprises whole or fragment of Protein Z.

9. The adapter system of claim 5, wherein the pAbBDs comprises whole or fragment of Protein G B1 domain.

10. The adapter system of any one of claims 6-9, wherein one or more photoreactive non-canonical amino acids are incorporated into each pAbBD.

11. The adapter system of any one of claims 6-9, wherein one or more photoreactive non-canonical amino acids are incorporated into each pAbBD, the photoreactive non-canonical amino acids selected from the group consisting of: azido-L-phenylalanine, benzophenone-alanine, benzoylphenylalanine, L-photoleucine, L-photomethionine, 3,4-difluorophenylalanine, 4,4,4,-trifluoro-L-valine, 5-fluoro-L-tryptophan, 5,5,5,-trifluoro-L-leucine, N^{ε}-(3-amino-5-azidobenzoylcarbonyl)-L-lysine, N^{ε}-(((3-((prop-2-yn-1-yloxy)methyl)-3H-diazirine-3-yl)methoxy)carbonyl)-L-lysine, and (Se—(N-(2-(3-(but-3-yn-1-yl)-3H-diazirine-3-yl)ethyl)propionamide)-3-yl-homoselenocysteine.

12. The adapter system of any one of claims 6-9, wherein one or more benzoylphenylalanine residues are incorporated into each pAbBD.

13. The adapter system of claim 5, wherein the pAbBD comprises a sequence having 80% or more sequence homology with either of SEQ ID NOs: 1 or 2 and has one or more photoreactive non-canonical amino acids substituted therein.

14. The adapter system of claim 5, wherein the pAbBD comprises SEQ ID NO: 1 wherein benzoylphenylalanine residues are substituted at F5, F13, L17, N23, Q32, K35, or D36, or at a combination thereof.

15. The adapter system of claim 5, wherein the pAbBD comprises SEQ ID NO: 1 wherein benzoylphenylalanine residues are substituted at least at Q32.

16. The adapter system of claim 5, wherein the pAbBD comprises SEQ ID NO: 2 wherein benzoylphenylalanine residues are substituted at A24, K28, or both.

17. The adapter system of claim 1, wherein the AbBD comprises one or more chemical-linking modules.

18. The adapter system of claim 17, wherein the chemical-linking modules are selected from the list consisting of: thiol, dibenzocyclooctyne, azide, alkyne, constrained alkyne, tetrazine, transcyclooctene, norbornene, and methylcyclopropene.

19. The adapter system of claim 1, wherein the AbBD comprises one or more peptide-linking modules.

20. The adapter system of claim 19, wherein the peptide-linking modules are selected from the list consisting of: intein, c-fos, c-jun, leucine zippers, peptide Velcro, SpyTag, SpyCatcher, sortase substrates, asparaginyl endoprotease substrates, subtiligase substrates, trypsiligase substrates, transglutaminase substrates.

21. The adapter system of claim 1, wherein each of the one or more epitope tags are the same type of epitope tag.

22. The adapter system of claim 1, comprising two or more different epitope tags.

23. The adapter system of claim 1, wherein the adapter system comprises at least two epitope tags.

24. The adapter system of claim 1, wherein the adapter system comprises at least three epitope tags.

25. The adapter system of claim 1, wherein the adapter system comprises at least four epitope tags.

26. The adapter system of claim 1, wherein the adapter system comprises at least two different epitope tags.

27. The adapter system of claim 1, wherein the epitope tags are of a type selected from: Arg-tag, Asp-tag, AU1, AU5, B-tag, Cys-tag, E, EE-tag, E2-tag, FLAG, 3×FLAG, HA, HAT, His-tag, HSV1, KT2, Lasso Tag, Myc, NorpA, OLLAAS, Phe-tag, Protein C tag, S-tag, SpyTag, Strep I, Strep II, Tag-100-tag, T7, Universal, V5, and VSV-G.

28. The adapter system of claim 1, wherein a linker is inserted between the epitope tags and/or between the AbBD and epitope tags.

29. The adapter system of claim 28, wherein the linker comprises one or more protease cleavage sites.

30. The adapter system of claim 1, wherein the AbBD comprises at least one cysteine residue, and wherein the at least one cysteine residue is rendered photocrosslinkable using a photoactive thiol-reactive agent.

31. The adapter system of claim 30, wherein the photoactive thiol-reactive agent is a maleimide reagent.

32. The adapter system of claim 30, wherein the photoactive thiol-reactive agent is 4-N-(maleimido)benzophenone.

33. An immunoglobulin-conjugate covalently linked to one or more epitope tags.

34. The epitope-modified immunoglobulin-conjugate of claim 33 wherein the immunoglobulin-conjugate comprises whole or fragment of a natural or recombinant IgG.

35. The epitope-modified immunoglobulin-conjugate of claim 34, wherein the one or more epitope tags are covalently bound to the whole or fragment of natural or recombinant IgG using photoreactive non-canonical amino acids.

36. The epitope-modified immunoglobulin-conjugate of claim 35, wherein the photoreactive non-canonical amino acids are selected from the group consisting of: azido-L-phenylalanine, benzophenone-alanine, benzoylphenylalanine, L-photoleucine, L-photomethionine, 3,4-difluorophenylalanine, 4,4,4,-trifluoro-L-valine, 5-fluoro-L-tryptophan, 5,5,5,-trifluoro-L-leucine, N^{ε}-(3-amino-5-azidobenzoylcarbonyl)-L-lysine, N^{ε}-(((3-((prop-2-yn-1-yloxy)methyl)-3H-diazirine-3-yl)methoxy)carbonyl)-L-lysine, and (Se—(N-(2-(3-(but-3-yn-1-yl)-3H-diazirine-3-yl)ethyl)propionamide)-3-yl-homoselenocysteine.

37. The epitope-modified immunoglobulin-conjugate of claim 35, wherein the photoreactive non-canonical amino acids are benzoylphenylalanine.

38. The epitope-modified immunoglobulin-conjugate of claim 35, wherein one or more protease cleavage sites are operably inserted between epitope tags and/or between the immunoglobulin-conjugate and the epitope tags.

39. An epitope-barcoded immunoglobulin-conjugate comprising a natural or recombinant IgG or fragment thereof, covalently crosslinked to one or more epitopes tags.

40. The epitope-barcoded immunoglobulin-conjugate of claim 39, wherein each epitope tag is operably linked to a protease cleavage site inserted between the epitope tags and/or between the epitope tag and IgG or IgG fragment.

41. A method of epitope-tagging an immunoglobulin or immunoglobulin fragment comprising the steps of:
providing at least one epitope tag having a crosslinker module; and
crosslinking the epitope tags to the immunoglobulin or immunoglobulin fragment.

42. The method of claim 41, wherein the crosslinker module is a photoreactive antibody binding domain (pAbBD), a click-chemistry module, or a peptide linking module.

43. The method of claim 41, wherein immunoglobulin or immunoglobulin fragment is whole or fragment of IgG, an F_{c}, a single-chain Fᵥ, an F_{ab}, F_{ab}′, Fᵥ, F(_{ab}′)₂, an affibody, monobody, anticalin, DARPin, or Knottin that has been fused or operably linked to IgG, F_{c}, or variant thereof.

44. The method of claim 41, wherein crosslinking the epitope tags to the immunoglobulin or immunoglobulin fragment comprises photo-crosslinking the epitope tags to the immunoglobulin or immunoglobulin fragment.

45. The method of claim 44, wherein the epitope tags comprise an antibody-binding domain (AbBD) selected from the list consisting of: F_{c} binding peptide; Protein A, Protein G, Protein L, Protein Z, or CD4; or a subdomain or functional fragment thereof.

46. The method of claim 44, wherein the AbBDs are photoreactive antibody-binding domains (pAbBDs) comprising one or more photoreactive non-canonical amino acids.

47. The method of claim 46, wherein the photoreactive non-canonical amino acids are selected from the group consisting of: azido-L-phenylalanine, benzophenone-alanine, benzoylphenylalanine, L-photoleucine, L-photomethionine, 3,4-difluorophenylalanine, 4,4,4,-trifluoro-L-valine, 5-fluoro-L-tryptophan, 5,5,5,-trifluoro-L-leucine, N^{ε}-(3-amino-5-azidobenzoylcarbonyl)-L-lysine, N^{ε}-(((3-((prop-2-yn-1-yloxy)methyl)-3H-diazirine-3-yl)methoxy)carbonyl)-L-lysine, and (Se—(N-(2-(3-(but-3-yn-1-yl)-3H-diazirine-3-yl)ethyl)propionamide)-3-yl-homoselenocysteine.

48. The method of claim 46, wherein the photoreactive non-canonical amino acids are benzoylphenylalanine.

49. The method of claim 46 wherein the pAbBD comprises a sequence having 80% or more sequence homology with either of SEQ ID NOs: 1 or 2 and has one or more photoreactive non-canonical amino acids substituted therein.

50. The method of claim 46, wherein the pAbBD comprises SEQ ID NO: 1 and wherein benzoylphenylalanine residues are substituted at F5, F13, L17, N23, Q32, K35, or D36, or at a combination thereof.

51. The method of claim 46, wherein the pAbBD comprises SEQ ID NO: 1 and wherein benzoylphenylalanine residues are substituted at least at Q32.

52. The method of claim 46, wherein the pAbBD comprises SEQ ID NO: 2 and wherein benzoylphenylalanine residues are substituted at A24, K28, or both.

53. The method of claim 46, wherein photo-crosslinking comprises mixing the epitope tags and immunoglobulin or immunoglobulin fragment and exposing the mixture to ultraviolet light.

54. The method of claim 53, wherein the ultraviolet light has a wavelength of about 365 nanometers.

55. The method of claim 41, wherein the epitope tags further comprise a protease cleavage site, such that upon crosslinking, the protease cleavage site is operably linked between the epitope tags, and/or between the epitope tags and immunoglobulin or immunoglobulin fragment.

56. The method of claim 41, wherein the crosslinker module comprises an AbBD comprising at least one cysteine residue, and wherein crosslinking comprises photoactivating a photoactive thiol-reactive agent.

57. The method of claim 56, wherein the photoactive thiol-reactive agent is a maleimide reagent.

58. The method of claim 57, wherein the maleimide reagent is 4-N-(maleimido)benzophenone.

59. A method of immunostaining a biological sample comprising exposing the sample to at least one epitope-modified immunoglobulin-conjugate of any one of claims 33-38.

60. The method of immunostaining of claim 59 further comprising a second step of exposing the sample to secondary-antibodies which are specific to the types of epitope tags bound to the epitope-modified immunoglobulin-conjugates.

61. The method of claim 59 comprising exposing the sample to at least two different epitope-modified immunoglobulin-conjugates.

62. The method of claim 59 comprising exposing the sample to at least three different epitope-modified immunoglobulin-conjugates.

63. The method of claim 59 comprising exposing the sample to at least four different epitope-modified immunoglobulin-conjugates.

64. A pull-down assay method comprising:
binding an epitope-conjugated antibody or epitope-conjugated antibody fragment to an antigen;
isolating the antigen using affinity resin designed to specifically bind an epitope on the epitope-conjugated antibody or epitope-conjugated antibody fragment.

65. The pull-down assay method of claim 64 further comprising the step of cleaving the epitopes from the epitope-conjugated antibody or epitope-conjugated antibody-fragment.

66. The pull-down assay method of claim 64, wherein cleaving the epitopes is accomplished by protease cleavage.

67. The method of any one of claims 64-66 further comprising:
(i) isolating the antigen using affinity resin that is designed to specifically bind a second epitope on the epitope-conjugated antibody or epitope-conjugated antibody fragment;
(ii) cleaving the second epitope from the epitope-conjugated antibody or epitope-conjugated antibody-fragment; and
(iii) optionally repeating (i) and (ii) with different epitopes and cleavage sites.

68. An extraction assay method comprising the steps of:
a. operably linking at least two epitope tags to an immunoglobulin or immunoglobulin fragment specific to a biomolecule be extracted from a mixture;
b. antibody-labeling the biomolecule to be extracted, the labeling forming a biomolecule-epitope-complex;
c. purifying the biomolecule-epitope-complex from the mixture using an affinity pulldown specific to one of the epitopes comprising the biomolecule-epitope-complex, then cleaving the epitope used in the purification step;
d. repeating step (c) until all epitope tags have been used in affinity pulldown and cleaved, leaving a purified biomolecule unbound to epitope tags.

69. The extraction assay method of claim 68 wherein the at least two epitope tags are bound to one another in series.

70. The extraction assay of method 68, wherein the at least two epitope tags are bound at the C-terminus of an antibody binding domain (AbBD).

71. The extraction assay of method 68, wherein the at least two epitope tags are bound at the N-terminus of an antibody binding domain (AbBD).

72. The extraction assay method of claim 68, wherein the at least two epitope tags are all different epitope tags.

73. The extraction assay method of claim 68, wherein the epitope tags are photocrosslinked to the antibody specific for the biomolecule to be extracted.

74. The extraction assay method of any one of claims 68-73, wherein the epitope tags are selected from the group consisting of: Arg-tag, Asp-tag, AU1, AU5, B-tag, Cys-tag, E, EE-tag, E2-tag, FLAG, 3×FLAG, HA, HAT, His-tag, HSV1, KT2, Lasso Tag, Myc, NorpA, OLLAAS, Phe-tag, Protein C tag, S-tag, SpyTag, Strep I, Strep II, Tag-100-tag, T7, Universal, V5, and VSV-G.

75. The extraction assay method of any one of claims 68-73, wherein the photoreactive non-canonical amino acids are selected from the group consisting of: azido-L-phenylalanine, benzophenone-alanine, benzoylphenylalanine, L-photoleucine, L-photomethionine, 3,4-difluorophenylalanine, 4,4,4,-trifluoro-L-valine, 5-fluoro-L-tryptophan, 5,5,5,-trifluoro-L-leucine, N^{ε}-(3-amino-5-azidobenzoylcarbonyl)-L-lysine, N^{ε}-(((3-((prop-2-yn-1-yloxy)methyl)-3H-diazirine-3-yl)methoxy)carbonyl)-L-lysine, and (Se—(N-(2-(3-(but-3-yn-1-yl)-3H-diazirine-3-yl)ethyl)propionamide)-3-yl-homoselenocysteine.


SEQUENCE LISTING (54 sequences)

SEQ ID NO: 1 - protein, 61 aa, Staphylococcus aureus
VDNKFNKEQQNAFYEILHLPNLNEEQRNAFIQSLKDDPSQSANLLAEAKKLNDAQAPKMRM

SEQ ID NO: 2 - protein, 56 aa, unidentified
MTFKLIINGKTLKGEITIEAVDAAEAEKIFKQYANDYGIDGEWTYDDATKTFTVTE

SEQ ID NO: 3 - protein, 8 aa, synthetic construct
DYKDDDDK

SEQ ID NO: 4 - protein, 6 aa, synthetic construct
HHHHHH

SEQ ID NO: 5 - protein, 9 aa, synthetic construct
YPYDVPDYA

SEQ ID NO: 6 - protein, 10 aa, synthetic construct
EQKLISEEDL

SEQ ID NO: 7 - protein, 13 aa, synthetic construct
GAPVPYPDPLEPR

SEQ ID NO: 8 - protein, 14 aa, synthetic construct
GKPIPNPLLGLDST

SEQ ID NO: 9 - protein, 15 aa, synthetic construct
KETAAAKFERQHMDS

SEQ ID NO: 10 - protein, 11 aa, synthetic construct
YTDIQMNRLGK

SEQ ID NO: 11 - protein, 9 aa, synthetic construct
NWSHPQFEK

SEQ ID NO: 12 - protein, 9 aa, synthetic construct
NANNPDWDF

SEQ ID NO: 13 - protein, 12 aa, synthetic construct
GVSSTSSDFRDR

SEQ ID NO: 14 - protein, 6 aa, synthetic construct
DTYRYI

SEQ ID NO: 15 - protein, 6 aa, synthetic construct
TDFYLK

SEQ ID NO: 16 - protein, 11 aa, synthetic construct
QPELAPEDPED

SEQ ID NO: 17 - protein, 6 aa, synthetic construct
QYPALT

SEQ ID NO: 18 - protein, 6 aa, synthetic construct
EYMPME

SEQ ID NO: 19 - protein, 6 aa, synthetic construct
APKPQP

SEQ ID NO: 20 - protein, 6 aa, synthetic construct
GGSGGS

SEQ ID NO: 21 - protein, 6 aa, synthetic construct
GGGGGG

SEQ ID NO: 22 - protein, 6 aa, synthetic construct
GGGGGS

SEQ ID NO: 23 - protein, 5 aa, synthetic construct
SSGSS

SEQ ID NO: 24 - protein, 6 aa, synthetic construct
GSGSGS

SEQ ID NO: 25 - protein, 7 aa, synthetic construct
AEAAAKA

SEQ ID NO: 26 - protein, 4 aa, synthetic construct
GGGS

SEQ ID NO: 27 - protein, 4 aa, synthetic construct
GSSG

SEQ ID NO: 28 - protein, 7 aa, synthetic construct
ENLYFQS

SEQ ID NO: 29 - protein, 6 aa, synthetic construct
LVPRGS

SEQ ID NO: 30 - protein, 4 aa, synthetic construct
IEGR

SEQ ID NO: 31 - protein, 5 aa, synthetic construct
  VARIANT 3: note = Any amino acid
LPXTG

SEQ ID NO: 32 - protein, 6 aa, synthetic construct
  VARIANT 6: note = Any amino acid
DDDDKX

SEQ ID NO: 33 - protein, 5 aa, synthetic construct
PRRAR

SEQ ID NO: 34 - protein, 54 aa, synthetic construct
TFKLIINGKTLKGEITIEAVDAEAEKIFKQYANDYGIDGEWTYDDATKTFTVTE

SEQ ID NO: 35 - protein, 56 aa, synthetic construct
TTYKLVINGKTLKGETTTEAVDAATAEKVFKQYAWDNGVTGEWTYDDATKTFTVTE

SEQ ID NO: 36 - protein, 56 aa, synthetic construct
TTYKLILNGKTLKGETTTEAVDAATAEKVFKQYAWDNGVTGEWTYDDATKTFTVTE

SEQ ID NO: 37 - protein, 56 aa, synthetic construct
TTYKLVINGKTLKGETTTKAVDAETAEKAFKQYAWDNGVTGVWTYDDATKTFTVTE

SEQ ID NO: 38 - protein, 58 aa, synthetic construct
VDNKINKEQQNAFYEILHLPNLNEEQRNAFIQSLKDDPSQSANLLAEAKKLNDAQAPK

SEQ ID NO: 39 - protein, 56 aa, synthetic construct
AQHDEAQQNAFYQVLNMPNLNADQRNGFIQSLKDDPSQSANVLGEAKKLNDSQAPK

SEQ ID NO: 40 - protein, 61 aa, synthetic construct
ADAQQNNFNKDQQSAFYEILNMPNLNEVQRNGFIQSLKDDPSQSTNVLGEAKKLNESQAPK

SEQ ID NO: 41 - protein, 58 aa, synthetic construct
ADNNFNKEQQNAFYEILNMPNLNEEQRNGFIQSLKDDPSQSANLLSEAKKLNESQAPK

SEQ ID NO: 42 - protein, 58 aa, synthetic construct [sequence omitted]

SEQ ID NO: 43 - protein, 58 aa, synthetic construct
ADNKFNKEQQNAFYEILHLPNLTEEQRNGFIQSLKDDPSVSKEILAEAKKLNDAQAPK

SEQ ID NO: 44 - protein, 34 aa, synthetic construct
FNMQCQRRFYEALHDPNLNEEQRNAKIKSIRDDC

SEQ ID NO: 45 - protein, 62 aa, synthetic construct
EVTIKVNLIFADGKIQTAEFKGTFEEATAEAYRYADLLAKVNGEYTADLEDGGNHMNIKFAG

SEQ ID NO: 46 - protein, 62 aa, synthetic construct
EVTIKVNLIFADGKTQTAEFKGTFEEATAEAYRYADLLAKVNGEYTADLEDGGYTINIKFAG

SEQ ID NO: 47 - protein, 62 aa, synthetic construct
EVTIKANLIFANGSTQTAEFKGTFEKATSEAYAYADTLKKDNGEYTVDVADKGYTLNIKFAG

SEQ ID NO: 48 - protein, 10 aa, synthetic construct
TWKTSRISIF

SEQ ID NO: 49 - protein, 10 aa, synthetic construct
FGRLVSSIRY

SEQ ID NO: 50 - protein, 12 aa, synthetic construct
EPIHRSTLTALL

SEQ ID NO: 51 - protein, 6 aa, synthetic construct
HWRGWV

SEQ ID NO: 52 - protein, 6 aa, synthetic construct
HYFKFD

SEQ ID NO: 53 - protein, 6 aa, synthetic construct
HFRRHL

SEQ ID NO: 54 - protein, 14 aa, synthetic construct
  VARIANT 14: note = Any amino acid
DCAWHLGELVWCTX